(19) United States
(12) Patent Application Publication
(10) Pub. No.: US 2022/0220512 A1
(43) Pub. Date: Jul. 14, 2022

(54) CARBON COMPOUND PRODUCTION IN PHOTOSYNTHETIC MICROORGANISMS

(71) Applicant: C3 BIOTECHNOLOGIES LIMITED, Lancaster (GB)

(72) Inventors: Nigel SCRUTTON, Lancaster (GB); Matthew FAULKNER, Lancaster (GB); Helen TOOGOOD, Lancaster (GB)

(21) Appl. No.: 17/596,346

(22) PCT Filed: Jun. 10, 2020
(86) PCT No.: PCT/EP2020/066092

§ 371 Date: Dec. 8, 2021

(30) Foreign Application Priority Data
Jun. 11, 2019 (GB) 1908317.9

(51) Int. Cl. C12P 7/06; C12N 1/20; C12P 7/62; C12N 9/16; C12N 9/00

(52) U.S. Cl. CPC C12P 7/065; C12N 1/20; C12P 7/62; C12Y 602/0102; C12Y 301/02014; C12N 9/93; C12N 9/16

(57) ABSTRACT

A method of upregulating the synthesis of pyruvate and/or a metabolite of pyruvate in a photosynthetic microorganism, a method for the photosynthetic production of a one, two, three or four carbon compounds, and a method for the production of ethanol from a photosynthetic microorganism are disclosed.

### FIELD OF THE INVENTION

The present invention relates to molecular biology and biotechnology and particularly, although not exclusively, to the production of pyruvate or metabolites of pyruvate in photosynthetic microorganisms.

### BACKGROUND

Photosynthetic microorganisms are one of the world's largest carbon sinks and are responsible for a large proportion of global carbon fixation by photosynthesis. These organisms are being explored as industrial chassis for carbon capture and conversion to useful products, in particular hydrocarbon biofuels (Lehtinen et al., 2018; Luan et al., 2015; Mikashi et al., 2016). The ability to take environmental CO₂ and convert it to useful biomass and fuels is highly desirable given the global targets around reducing CO₂ emissions (Gao et al., 2012; Kanno et al., 2017). This process could potentially create a cyclical green CO₂ economy for processes such as power generation which currently consume large amounts of fossil fuels (Yoshikawa et al., 2017).

Cyanobacteria have a widely reported natural ability to convert CO₂ into ethanol in small amounts (Luan et al., 2015; Gao et al., 2012; Yoshikawa et al., 2017; Lawrence et al., 2014). This is because the photosynthetic conversion of CO₂ through to pyruvate does not result in high concentrations of, or high levels of metabolic flux through, pyruvate (Gao et al., 2012; Carrieri et al., 2010; Oliver and Atsumi, 2015, Subashchandrabose et al., 2011). Therefore recent studies have tried to increase cyanobacterial ethanol production under non-photosynthetic growth conditions (Luan et al., 2015; Gao et al., 2012; Yoshikawa et al., 2017; Lawrence et al., 2014). The current record titre of photosynthetic cyanobacterial ethanol production is 5.5 g/L after 26 days, or 2 g/L/day (Lehtinen et al., 2018). This was obtained by genetic manipulation of Synechocystis to incorporate the genes encoding pyruvate decarboxylase and alcohol dehydrogenase, effectively generating an alternative ethanol production pathway (Lehtinen et al., 2018). A range of other techniques have not been able to achieve higher yields, most likely due to the lack of flux from fixed carbon to pyruvate (Dexter et al., 2015). However these methods generate titres considerably lower than the current ‘gold standard’ process of yeast anaerobic fermentation of biomass to ethanol (˜4-5 g/L/h up to titres of ˜100-120 g/L in ˜24 hours) (Mohd Azhar et al., 2017). This requires a large amount of feedstock material (˜200-500 g/L sugars), contributing to the significant costs of the process.

The current cyanobacteria ethanol productivity values are too low to be support an economically viable distillation process to recover and purify the ethanol. Given the significant reduction in feedstock costs and economic advantages of a sustainable carbon capture solution, an economically viable process could be devised if the minimum threshold of 10% ethanol can be generated in cyanobacteria (Kanno et al., 2017).

A recent review into the use of cyanobacteria as biochemical ‘factories’ identified a major bottleneck to increasing chemical titres is an imbalance in carbon flux distribution, where typically only 5-10% of carbon goes through pyruvate to the terpenoid and fatty acid biosynthesis pathways (Carrieri, D. et al., 2010; Gao et al., 2016; Gao et al, 2012; Oliver and Atsumi, 2015; Subashchandrabose et al., 2011). This is true in the case of much more complex anabolic pathways leading to a whole range of products based on pyruvate, whose productivity is pyruvate flux limited, such as terpenoids via the methylerythritol 4-phosphate (MEP) pathway (Pattanaik and Lindberg, 2015). In the case of fatty acid, polyhydroxybutyrate (PHB) and ethanol biosynthesis, key intermediates are pyruvate and acetyl-CoA (FIG. 1), so increasing their intracellular concentrations is the most direct way to increase ethanol titres. The native Synechocystis route to ethanol production from acetyl-CoA requires acetaldehyde dehydrogenase (AldDH) and alcohol dehydrognase slr1192 (aadhA; FIG. 1) in the latter two steps (Vidal et al., 2009). In addition, engineered Synechocystis strains have been developed to increase ethanol titres by the addition of the genes pyruvate decarboxylase (PDC) and alcohol dehydrogenase ll from Zymomonas mobilis (Gao et al, 2012). This non-native pathway is an alternative 2-step route from pyruvate to ethanol (Gao et al, 2012). Therefore both the native and established recombinant route to ethanol production in Synechocystis require pyruvate production as an intermediate step. It therefore follows that any route in Synechocystis that leads to increasing ethanol titres must by definition involve increasing carbon flux through pyruvate (Vidal et al., 2009).

Previous research has used a process for the enhanced production of exopolysaccharide in microalgae or cyanobacteria which involves using an increased irradiance stimulus, using two peaks of red and blue light (WO2012101495A2). No work was conducted with a single peak of blue light.

Blue light has not previously been considered as an option to upregulate the synthesis of carbon compounds such as ethanol or pyruvate. Cyanobacteria use blue light less efficiently for photosynthesis than most eukaryotic phototrophs. Furthermore, blue light has long been known to decrease the photosynthetic efficiency of cyanobacteria via photo bleaching and cell death at high intensities (Hirosawa, 1984; Luimstra et al, 2018).

The present disclosure has been devised in light of the above considerations.

### SUMMARY OF THE INVENTION

The inventors have unexpectedly discovered that blue light can upregulate the synthesis of carbon compounds, such as pyruvate and metabolites of pyruvate, in photosynthetic microorganisms. This discovery provides the basis for methods of upregulating the synthesis of pyruvate and/or metabolites of pyruvate in a photosynthetic microorganism, and for methods for the photosynthetic production in photosynthetic microorganisms of pyruvate and/or metabolites of pyruvate, which may be one, two, three or four carbon containing compounds.

In one aspect of the present invention a method of upregulating the synthesis of pyruvate and/or a metabolite of pyruvate in a photosynthetic microorganism is provided, the method comprising culturing the photosynthetic microorganism under conditions suitable for photosynthesis wherein the culture is irradiated with light having a wavelength between 380 nm and 500 nm.

The method may further comprise the step of isolating said pyruvate or metabolite of pyruvate from the photosynthetic microorganism.

The metabolite of pyruvate may be selected from one of ethanol, acetyl-CoA, alanine, butanol, propane, acetate, butyrate or lactate. In some embodiments, the metabolite of pyruvate is not butyric acid or propane.

The method may be part of a method for the production of more complex products, such as terpenoids, via the native anabolic pathways from pyruvate, such as the MEP pathway.

In one embodiment, the method is a method of upregulating the synthesis of pyruvate and/or ethanol in a photosynthetic microorganism, the method comprising culturing the photosynthetic microorganism under conditions suitable for photosynthesis wherein the culture is irradiated with light having a wavelength between 380 nm and 500 nm, the method further comprising the step of isolating said pyruvate or ethanol from the photosynthetic microorganism or from the culture.

In another aspect of the present invention a method for the photosynthetic production of a one, two, three or four carbon compound is provided, the method comprising culturing a photosynthetic microorganism under conditions suitable for photosynthesis wherein the culture is irradiated with light having a wavelength between 380 nm and 500 nm, and isolating the one, two, three or four carbon compound.

The one, two, three or four carbon compound may be pyruvate or a metabolite of pyruvate. The metabolite of pyruvate may be selected from one of ethanol, acetyl-CoA, alanine, butanol, propane, acetate, butyrate or lactate. In some embodiments, the metabolite of pyruvate is not butyric acid or propane.

The method may be for the production and/or isolation of two or three carbon compounds, such as pyruvate or ethanol.

In some embodiments, a method disclosed herein is not a method for the blue light-dependent decarboxylation of fatty acids to alkanes or alkenes. For example, the method is optionally not a method of driving, activating, or upregulating the activity of an alkane producing enzyme such as a photodecarboxylase, e.g. Chlorella vulgaris fatty acid photodecarboxylase (CvFAP) or a homologue thereof.

The culture may be irradiated with light having a proportion of light having a wavelength between 380 nm and 500 nm that is greater than that normally found in white light. As such, the culture may be irradiated with light having a proportion of light having a wavelength between 380 nm and 500 nm that is greater than 35%. The proportion may be greater than or equal to one of 35% (e.g. greater than or equal to one of 35%, 36%, 37%, 38%, 39% or more), greater than or equal to one of 40% (e.g. greater than or equal to one of 40%, 41%, 42%, 43%, 44% or more), greater than or equal to one of 45% (e.g. greater than or equal to one of 45%, 46%, 47%, 48%, 49%), or greater than or equal to one of 50% (e.g. greater than or equal to one of 50%, 51%, 52%, 53%, 54%, 55%, or more).

Accordingly, the culture may be irradiated with light having a proportion of light having a wavelength between 501 nm and 800 nm that is less than the proportion normally found in white light. As such, the culture may be irradiated with light having a proportion of light having a wavelength between 501 nm and 800 nm that is less than 50%, or less than one of 45%, 40%, 35%, 30%, 25%, 20%, 15%, 10%, 5%, 4%, 3%, 2%, or 1%.

Accordingly, of the light in the wavelength range 380 nm to 800 nm applied to the culture, greater than one of 35%, 40%, 45%, 50%, 55%, 60%, 65%, 70%, 80%, 85%, 90%, or 95% may be light having a wavelength between 380 nm and 500 nm and/or less than one of 65%, 60%, 55%, 50%, 45%, 40%, 35%, 30%, 25%, 20%, 15%, 10%, or 5% may be light having a wavelength between 501 nm and 800 nm.

Accordingly, the culture may be irradiated with a single peak of blue light. The single peak of blue light may have a peak of light within the spectrum of wavelength between 380 nm and 500 nm. The single peak of blue light may have a peak of light within the spectrum of wavelength between one of 390 nm and 500 nm, 390 nm and 490 nm, 390 nm and 480 nm, 390 nm and 470 nm, 400 nm and 500 nm, 400 nm and 490 nm, 400 nm and 480 nm, 400 nm and 470 nm, 410 nm and 500 nm, 410 nm and 490 nm, 410 nm and 480 nm, 410 nm and 470 nm, 420 nm and 500 nm, 420 nm and 490 nm, 420 nm and 480 nm, 420 nm and 470 nm, 430 nm and 500 nm, 430 nm and 490 nm, 430 nm and 480 nm, 430 nm and 470 nm, 440 nm and 500 nm, 440 nm and 490 nm, 440 nm and 480 nm, 440 nm and 470 nm, 450 nm and 500 nm, 450 nm and 490 nm, 450 nm and 480 nm, 450 nm and 470 nm.

The light having a wavelength between 380 nm and 500 nm (‘blue light’) may have a photon dose greater than 10 μmol m⁻²s⁻¹. Optionally the photon dose is less than one of 3000, 2000, 1000, 900, 800, or 700 μmol m⁻²s⁻¹.

In some embodiments, the light having a wavelength between 380 nm and 500 nm (‘blue light’) may have a photon dose greater than 20, 30, 40, 50, 60, 70, 80, 90, 100, 150, 200, 250, 300, 350, or 400 μmol m⁻² s⁻¹.

In some embodiments, the light having a wavelength between 380 nm and 500 nm (‘blue light’) may have a photon dose which is less than one of 3000, 2500, 2000, 1500, 1000, 900, 800, or 700 μmol m⁻²s⁻¹.

The culture may be irradiated with light having a wavelength between 380 nm and 500 nm for a period of time sufficient to upregulate the synthesis of pyruvate and/or a metabolite of pyruvate in the photosynthetic microorganism. For example, the culture may be irradiated with light having a wavelength between 380 nm and 500 nm, for one of at least at least 5, 10, 15, 30, 45, or 60 minutes, or one of at least 1, 2, 3, 4, 5, 6, 7, 8, 9, 10, 11, 12, 13, 14, 15, 16, 17, 18, 19, 20, 21, 22, 23, or 24 hours, or more.

In accordance with the above, the culture may be irradiated with light from a light source that emits light having a proportion of light having a wavelength between 380 nm and 500 nm that is greater than one of 35%, 40%, 45%, or 50%. The culture may be irradiated with light from a light source that emits said light having a wavelength between 380 nm and 500 nm at a photon dose greater than 10 μmol m⁻²s⁻¹, and optionally less than one of 3000, 2000 or 1000 μmol m⁻²s⁻¹.

The culture may be irradiated with light having a wavelength between 380 nm and 500 nm for a period of time sufficient to produce ethanol at a concentration of one of at least 2% (w/v), 3% (w/v), 4% (w/v), 5% (w/v), 6% (w/v), 7% (w/v), 8% (w/v), 9% (w/v), 10% (w/v), or more, in the culture.

The culture may be irradiated with light having a wavelength between 380 nm and 500 nm for a period of time sufficient to produce ethanol at over one of 0.1 g/L/h, 0.2 g/L/h, 0.3 g/L/h, 0.4 g/L/h, 0.5 g/L/h, 0.6 g/L/h, 0.7 g/L/h, 0.8 g/L/h, 0.9 g/L/h, 1.0 g/L/h, 1.1 g/L/h, 1.2 g/L/h, 1.3 g/L/h, 1.4 g/L/h, 1.5 g/L/h, 1.6 g/L/h, 1.7 g/L/h, 1.8 g/L/h, 1.9 g/L/h, 2.0 g/L/h, 2.5 g/L/h, 3.0 g/L/h, 3.5 g/L/h, 4.0 g/L/h, 4.5 g/L/h, 5.0 g/L/h, or 5.5 g/L/h.

The culture may be irradiated with light having a wavelength between 380 nm and 500 nm, at a photon dose sufficient to produce ethanol at a concentration of at least one of 2% (w/v), 3% (w/v), 4% (w/v), 5% (w/v), 6% (w/v), 7% (w/v), 8% (w/v), 9% (w/v), 10% (w/v), or more, in the culture.

The culture may be irradiated with light having a wavelength between 380 nm and 500 nm, at a photon dose sufficient to produce ethanol at a concentration of one of at least 2% (w/v), 3% (w/v), 4% (w/v), 5% (w/v), 6% (w/v), 7% (w/v), 8% (w/v), 9% (w/v), 10% (w/v), or more, in the culture.

The culture may be irradiated with light having a wavelength between 380 nm and 500 nm, at a photon dose sufficient to produce ethanol at over one of 0.1 g/L/h, 0.2 g/L/h, 0.3 g/L/h, 0.4 g/L/h, 0.5 g/L/h, 0.6 g/L/h, 0.7 g/L/h, 0.8 g/L/h, 0.9 g/L/h, 1.0 g/L/h, 1.1 g/L/h, 1.2 g/L/h, 1.3 g/L/h, 1.4 g/L/h, 1.5 g/L/h, 1.6 g/L/h, 1.7 g/L/h, 1.8 g/L/h, 1.9 g/L/h, 2.0 g/L/h, 2.5 g/L/h, 3.0 g/L/h, 3.5 g/L/h, 4.0 g/L/h, 4.5 g/L/h, 5.0 g/L/h, or 5.5 g/L/h.

In some embodiments the method may comprise culturing the photosynthetic microorganism under white light irradiation or under non-blue light irradiation for a period of time, optionally during which the photosynthetic microorganism may grow, before light conditions are changed to irradiation with light having a wavelength between 380 nm and 500 nm.

As such the method may comprise culturing the photosynthetic microorganism under standard growth conditions, prior to being cultured under irradiation with light having a wavelength between 380 nm and 500 nm.

The photosynthetic microorganism may be grown in a first vessel under white light or non-blue light irradiation, before being transferred to a second vessel to be cultured under irradiation with light having a wavelength between 380 nm and 500 nm.

The photosynthetic microorganism may be grown to a high cell density, e.g. determined by an OD₆₈₀ measurement of over 0.5, before commencing irradiation with light having a wavelength between 380 nm and 500 nm.

As such, the photosynthetic microorganism culture may be irradiated with white light or non-blue light conditions until the photosynthetic microorganism is grown to a high cell density, e.g. determined by an OD₆₈₀ measurement of over 0.6, 0.7, 0.8, 0.9, 1.0, or 1.1, before commencing irradiation with light having a wavelength between 380 nm and 500 nm.

The photosynthetic microorganism may be grown in batch culture or in continuous culture.

In some embodiments, following irradiation with light having a wavelength between 380 nm and 500 nm, the photosynthetic microorganism fraction and liquid fraction are separated, and the product is isolated from the liquid fraction. The product (e.g. pyruvate, ethanol, pyruvate metabolite or one, two, three or four carbon containing compound) may be isolated through distillation or other separation techniques, e.g. chromatographic separation. The photosynthetic microorganism fraction may be used for animal feed or as a fertiliser or as feedstock for another biotechnology process.

In some embodiments, the photosynthetic microorganism may be modified, e.g. genetically modified, to reduce fatty acid synthesis. The photosynthetic microorganism may be modified to:
a) increase the carbon flux from pyruvate to ethanol,
b) reduce the conversion of acetyl-CoA to malonyl-ACP,
c) reduce acyl-acyl carrier protein synthase activity,
d) increase the conversion of butyryl-ACP to butyric acid,
e) increase acyl-ACP-thioesterase expression,
f) heterologously express an acyl-ACP-thioesterase gene,
g) heterologously express the thioesterase gene tes4 from Bacteroides fragilis, and/or
h) express enzymes to redirect carbon flux from pyruvate to other anabolic pathways.

In some embodiments the photosynthetic microorganism may be modified to (i) increase the carbon flux from pyruvate to ethanol and/or (ii) reduce the conversion of acetyl-CoA to malonyl-ACP. In some embodiments the acyl-acyl carrier protein synthase activity is reduced or knocked out in the photosynthetic microorganism.

In another aspect of the present invention, a method for the production of ethanol from a photosynthetic microorganism is provided, the method comprising culturing the photosynthetic microorganism under conditions suitable for photosynthesis, wherein the photosynthetic microorganism has been modified to reduce fatty acid synthesis.

The use of a photosynthetic microorganism which has been modified to reduce fatty acid synthesis, for the production of ethanol is also provided.

The method or photosynthetic microorganism may be capable of producing a concentration of ethanol in culture of at least one of 2% (w/v), 3% (w/v), 4% (w/v), 5% (w/v), 6% (w/v), 7% (w/v), 8% (w/v), 9% (w/v), or 10% (w/v).

The method or photosynthetic microorganism may be capable of producing a concentration of ethanol in culture of at least one of over 0.1 g/L/h, 0.2 g/L/h, 0.3 g/L/h, 0.4 g/L/h, 0.5 g/L/h, 0.6 g/L/h, 0.7 g/L/h, 0.8 g/L/h, 0.9 g/L/h, 1.0 g/L/h, 1.1 g/L/h, 1.2 g/L/h, 1.3 g/L/h, 1.4 g/L/h, 1.5 g/L/h, 1.6 g/L/h, 1.7 g/L/h, 1.8 g/L/h, 1.9 g/L/h, 2.0 g/L/h, 2.5 g/L/h, 3.0 g/L/h, 3.5 g/L/h, 4.0 g/L/h, 4.5 g/L/h, 5.0 g/L/h, or 5.5 g/L/h.

The photosynthetic microorganism may be modified to:
a) increase the carbon flux from pyruvate to ethanol,
b) reduce the conversion of acetyl-CoA to malonyl-ACP,
c) reduce acyl-acyl carrier protein synthase activity,
d) increase the conversion of butyryl-ACP to butyric acid,
e) increase acyl-ACP-thioesterase expression,
f) heterologously express an acyl-ACP-thioesterase gene, and/or
g) heterologously express the thioesterase gene tes4 from Bacteroides fragilis.

In some embodiments the photosynthetic microorganism may be modified to (i) increase the carbon flux from pyruvate to ethanol and/or (ii) reduce the conversion of acetyl-CoA to malonyl-ACP. In some embodiments the acyl-acyl carrier protein synthase activity is reduced or knocked out in the photosynthetic microorganism.

In any aspect of the present invention the photosynthetic microorganism may be a photosynthetic cyanobacterium or microalgae.

The photosynthetic microorganism may be a cyanobacterium selected from the class Cyanophyceae Cyanophyceae, Chroobacteria, Hormogoneae, or Gloeobacteria, the family Merismopediaceae, Prochloraceae or Prochlorotrichaceae, the genera Halospirulina, Planktothricoides, Prochlorococcus, Prochloron, or Prochlorothrix

In some embodiments, the photosynthetic microorganism is selected from Acaryochloris sp., Acaryochloris marina, Chamaesiphon minutus, Crocosphaera watsonii, Cyanobacterium aponinum, Cyanobacterium stanieri, Cyanobium gracile, Cyanobium sp., Cyanothece sp., Dactylococcopsis salina, Geminocystis herdmanii, Gloeobacter violaceus, Gloeocapsa sp., Gloeocapsa sp., Halothece sp., Microcystis aeruginosa, Prochlorococcus marinus, Prochloron didemni, Synechococcus sp., Synechococcus elongates, or Thermosynechococcus elongates.

The photosynthetic microorganism may be a cyanobacterium from the class Cyanophyceae.

The photosynthetic microorganism may be a Synechocystis species.

The photosynthetic microorganism may be a microalgal microorganism selected from the Rhodophyta, Chrysophyceae, Phaeophyceae, or the Chlorophyta group.

In some embodiments, the photosynthetic microorganism is selected from Arthrospira sp., Chlamydomonas reinhardtii, Chlorella sp., Chlorella vulgaris, Dunaliella salina, Haematococcus pluvialis, Odontella aurita, Porphyridium cruentum, Isochrysis galbana, Phaedactylum tricomutum, Lyngbya majuscule, Scenedesmus sp., Schizochytrium sp., Crypthecodinium cohnii., Nannochloropsis oculata., and Nannochloropsis sp.

Photosynthetic microorganisms may be naturally occurring (wild type) or genetically modified, e.g. to increase the carbon flux from pyruvate to ethanol and/or to reduce the conversion of acetyl-CoA to malonyl-ACP.

In some embodiments, acyl-acyl carrier protein synthase activity is reduced or knocked out in the photosynthetic microorganism. In some embodiments, native acyl-acyl carrier protein synthase activity is reduced or knocked out in the photosynthetic microorganism. In some embodiments, the photosynthetic microorganism has been modified to increase the conversion of butyryl-ACP to butyric acid. In some embodiments, the photosynthetic microorganism has been modified to increase acyl-ACP-thioesterase expression. In some embodiments, the photosynthetic microorganism has been modified to heterologously express an acyl-ACP-thioesterase gene. In some embodiments, the photosynthetic microorganism has been modified to heterologously express the thioesterase gene tes4, e.g. from Bacteroides fragilis. In some embodiments, the photosynthetic microorganism has been modified to reduce native acyl-acyl carrier protein synthase activity, and to heterologously express the thioesterase gene tes4, e.g. from Bacteroides fragilis.

In any aspect of the present invention the culture may be supplied with an additional carbon source in addition to atmospheric carbon dioxide. Examples of a suitable carbon supply include carbon dioxide, e.g. as a gas passed through the culture, sugar(s), or NaHCO₃ which may be added to the culture.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Synechocystis carbon flux through pyruvate and acetyl-CoA, including native ethanol biosynthesis. Enzymes: AlaDH=alanine dehydrogenase; ldh=lactate dehydrogenase; me=malic enzyme; pta=phosphotransacetylase; acs=acetyl-coenzyme A synthetase; ackA=acetate kinase; AldDH=acetaldehyde dehydrogenase and aadhA=alcohol dehydrognase slr1192.6 PHB=polyhydroxybutyrate; CBB=Calvin—Benson—Bassham cycle.

FIG. 2. Proposed route of ethanol production up-regulation in Synechocystis sp. PCC 6803. 3-PGA=3-phosphoglycerate; CO₂=carbon dioxide; aas=acyl-acyl carrier protein synthase; Tes4=acyl-ACP thioesterase 4; CBB=Calvin—Benson—Bassham cycle.

FIG. 3. Pyruvate metabolism and the generation of pyruvate metabolites.

FIG. 4. Growth of Synechocystis sp. PCC 6803 a) wild-type, b) Δaas and c) plY845 strains.

FIG. 5. Visualisation of Synechocystis sp. PCC 6803 a) wild-type, b) Δaas and c) plY845 strain growth after 24 hours.

FIG. 6. Visualisation of Synechocystis sp. PCC 6803 a) wild-type, b) Δaas and c) plY845 strain growth after 48 hours.

FIG. 7. Ethanol concentration during growth of Synechocystis sp. a) PCC 6803 wild-type, b) Δaas and c) plY845 strains.

FIG. 8. Ethanol production by Synechocystis plY845 in white light.

FIG. 9. Ethanol production by Synechocystis plY845 under blue light, with NaHCO₃ supplementation.

FIG. 10. NMR spectra of solvent extracted samples from a photobioreactor run. Aqueous samples of a) culture extract, b) condensate and c) 1% (w/v) ethanol were extracted into deuterated chloroform. The shift to the right observed of the ethanol peaks in the condenser spectra is likely due to the high concentration of ethanol compared to the other samples.

FIG. 11. Ethanol production by Synechocystis plY845 under blue light, with NaHCO₃ supplementation and a higher initial cell density.

FIG. 12. Ethanol production by Synechocystis plY845 under blue light, without extra NaHCO₃ supplementation.

FIG. 13. Production of ethanol by Synechocystis cultures grown under white light.

FIG. 14. Production of ethanol by Synechocystis cultures grown under white vs blue light with bicarbonate feeding. The white light cultures were grown under white light for the whole 50 hour experiment. Blue light cultures were grown under white light between 0 and 20.6 hours, then grown under 750 μE m⁻²s⁻¹ blue light between 20.6 and 20.8 hours, and then grown under 500 μE m⁻²s⁻¹ blue light between 20.8 and 50 hours

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present disclosure is based on the identification by the inventors that photosynthetic microorganisms can be utilised in novel methods for the production of pyruvate or metabolites of pyruvate. The process described here is based on high levels of metabolic flux through pyruvate driven by photosynthesis, and provides a basis for industrial scale production of one, two, three or four carbon compounds from photosynthetic microorganisms, optionally utilising waste carbon dioxide (CO₂) as the carbon source. More complex metabolites and products such as terpenoids can be made, optionally from waste CO₂, via the native anabolic pathways such as the MEP pathway using pyruvate as a precursor, that also depend on flux through pyruvate.

The inventors found that blue light can be utilised to upregulate the synthesis of pyruvate and ethanol in a photosynthetic microorganism. This result was unexpected as blue light is known to be harmful to such organisms; whilst investigating blue light for use in activation of an enzyme of interest in the study of propane and butyrate/butyric acid production from microorganisms the inventors surprisingly found that ethanol production was up-regulated. Further research led the inventors to the discovery that blue light can upregulate the metabolic flux through pyruvate, providing a basis for increasing production of pyruvate and metabolites of pyruvate in photosynthetic microorganisms.

The inventors also found that photosynthetic microorganisms can be genetically manipulated to improve their ability to produce ethanol, by reducing metabolic flux towards fatty acid synthesis. When combined with the application of blue light this led to even higher levels of ethanol production.

Photosynthetic Microorganisms

There are a wide range of photosynthetic microorganisms that have great potential in biotechnology applications. One reason for this interest is their ability to fix carbon dioxide from the environment, through photosynthesis, and produce compounds of interest, such as ethanol.

Cyanobacteria and microalgae are two groups of photosynthetic microorganisms.

Cyanobacteria hold significant potential as industrial biotechnology platforms for the production of a wide variety of bio-products ranging from biofuels such as hydrogen, alcohols, isoprenoids, and terpenoids to high-value bioactive and recombinant proteins (Al-Haj et al., 2016).

At present, there is some disagreement with regards to the classification of cyanobacteria. The cyanobacteria (Cyanophyta) are also named under the Botanical Code, and the dual nomenclature system causes considerable confusion (Oren, 2004). However, classes of cyanobacteria include: Cyanophyceae, Chroobacteria, Hormogoneae, and Gloeobacteria. Orders include: Synechococcales, Chroococcales, Gloeobacterales, Nostocales, Oscillatoriales, Pleurocapsales, and Stigonematales. Families include: Merismopediaceae, Prochloraceae and Prochlorotrichaceae. Genera include Halospirulina, Planktothricoides, Prochlorococcus, Prochloron, and Prochlorothrix.

In one embodiment, the photosynthetic microorganism is a cyanobacterium. The photosynthetic microorganism may be a cyanobacterium from the class Cyanophyceae Cyanophyceae, Chroobacteria, Hormogoneae, or Gloeobacteria. The cyanobacterium may be from the family Merismopediaceae, Prochloraceae or Prochlorotrichaceae. The cyanobacterium may be from the genera Halospirulina, Planktothricoides, Prochlorococcus, Prochloron, or Prochlorothrix.

The photosynthetic microorganism may be one of Acaryochloris sp., Acaryochloris marina, Chamaesiphon minutus, Crocosphaera watsonii, Cyanobacterium aponinum, Cyanobacterium stanieri, Cyanobium gracile, Cyanobium sp., Cyanothece sp., Dactylococcopsis salina, Geminocystis herdmanii, Gloeobacter violaceus, Gloeocapsa sp., Gloeocapsa sp., Halothece sp., Microcystis aeruginosa, Prochlorococcus marinus, Prochloron didemni, Synechococcus sp., Synechococcus elongatus, Synechocystis sp., Synechocystis sp. PCC 6803, Synechocystis sp. PCC 6714, Synechocystis crassa, Synechocystis salina, Synechocystis maior, or Thermosynechococcus elongatus.

Another group of photosynthetic microorganisms are microalgae. Like cyanobacteria, microalgae are also well studied in the field of biofuels. Microalgae are essentially microscopic algae.

Microalgae are traditionally divided according to their light-harvesting photosynthetic pigments: Rhodophyta (red algae), Chrysophyceae (golden algae), Phaeophyceae (brown algae), and Chlorophyta (green algae).

In some embodiments, the photosynthetic microorganism is a microalgal microorganism from the Rhodophyta, Chrysophyceae, Phaeophyceae, or the Chlorophyta group.

The microalgal microorganism may be one of is Arthrospira sp., Chlamydomonas reinhardtii, Chlorella sp., Chlorella vulgaris, Dunaliella salina, Haematococcus pluvialis, Odontella aurita, Porphyridium cruentum, Isochrysis galbana, Phaedactylum tricomutum, Lyngbya majuscule, Scenedesmus sp., Schizochytrium sp., Crypthecodinium cohnii., Nannochloropsis oculata., and Nannochloropsis sp.

Culture of Photosynthetic Microorganisms

Photosynthetic microorganisms can be cultured in flasks, bioreactors, photobioreactors, open ponds, raceway ponds, or any other vessel which can hold media and allow light entry. In one embodiment of the disclosure, cyanobacteria are grown in a bioreactor. Methods according to the present invention may be performed on industrial scale, e.g. in vessels that hold from 1 litre to 1000 litres (e.g. 10, 20, 30, 40, 50, 60, 70, 80, 90, 100, 150, 200, 250, 300, 350, 400, 450, 500, 550, 600, 650, 700, 750, 800, 850, 900, 950, 1000 litres) or more of culture.

Photosynthetic microorganisms can be cultured in a number of ways. Typically, photosynthetic organisms are cultured in conditions suitable for photosynthesis. Such conditions require light, water, and a carbon source to be available. Further conditions may improve growth, biomass and compound yield, but the essential components for photosynthesis in photosynthetic microorganisms are light, water, and a carbon source (Ruffing, 2011).

Photosynthetic microorganisms may be grown in batch cultures or in continuous cultures, according to techniques well known to those of ordinary skill in the art, e.g. see Pors et al., 2010 and Fernandes et al., 2015.

The culture or fermentation may be performed in a bioreactor provided with an appropriate supply of nutrients, air/oxygen, carbon dioxide, and/or growth factors. Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, light, dissolved CO₂, and agitation within the vessel such that optimum conditions are provided for the cells being cultured. Following culture, in order to isolate the product of interest it may be necessary to separate the cells from culture medium. Any suitable method for separating the product of interest from cells known in the art may be used. For example, if the product is secreted from the cells, the cells may be separated from the culture media that contains the secreted product of interest by centrifugation.

If the product of interest collects within the cell, product isolation may comprise centrifugation to separate cells from cell culture medium, treatment of the cell pellet with a lysis buffer, and cell disruption e.g. by sonication, rapid freeze-thaw or osmotic lysis.

It may then be desirable to isolate the product of interest from the supernatant or nutrient medium, e.g. by distillation or by chromatographic separation.

Carbon Source

In the wild, photosynthetic microorganisms tend to utilise atmospheric carbon dioxide as their carbon source.

In some embodiments of this invention, photosynthetic microorganisms utilise atmospheric carbon dioxide as their carbon source. In some embodiments a carbon supply is provided in addition to atmospheric carbon dioxide. Suitable additional carbon supplies may be provided by the addition of sugar, NaHCO₃, waste glycerol, pre-treated food waste, pre-treated plant waste, pre-treated seaweed, or supplemental CO₂ to the culture.

The additional carbon may be supplied as a gas, liquid or a solid.

The additional carbon supply may be added continuously or at selected times during the culture or growth cycle of the photosynthetic microorganism.

In one embodiment, the additional carbon supply is CO₂. Carbon dioxide (CO₂) is produced as a by-product of many industrial processes such as oil and gas production, cement production, iron and steel production, and electricity generation, as well as many others. This carbon dioxide is typically released into the environment through the combustion of fuels. However, it can be captured, separated, isolated and stored. This disclosure provides methods of utilising this waste carbon dioxide in a process of photosynthetic carbon fixation producing products of value and industrial application.

Light Conditions

Light is required for photosynthesis. Light is electromagnetic radiation within a certain portion of the electromagnetic spectrum. White light is typically used to grow photosynthetic microorganisms, and is a combination of photons of different wavelengths in the electromagnetic spectrum.

Electromagnetic radiation with a wavelength between 380 nm and 760 nm is normally perceived as visible light by humans. Other wavelengths, especially near infrared (longer than 760 nm) and ultraviolet (shorter than 380 nm) are also sometimes referred to as light, especially when visibility to humans is not relevant.

White light is a combination of lights of different wavelengths in the visible spectrum. White light is normally a complete or substantially complete mixture of all of the wavelengths of the visible spectrum (between 380 nm and 760 nm). White light normally contains light of wavelengths between 380 nm and 760 nm in approximately equal proportions. One example of white light is ordinary daylight. White light can be generated by a variety of sources, such as the sun. Fluorescent light bulbs and white LEDs will normally produce white light. A typical domestic fluorescent light bulb produces a photon dose of 130 μmol m⁻²s⁻¹ at a distance of 1 cm. One typical white light photon dose which can be used for photosynthesis in photosynthetic microorganisms is 30-60 μmol m⁻²s⁻¹. Other light bulbs, such as the incandescent lamp, do not normally produce white light but produce light of long wavelengths in the yellow to red range.

Blue light has a shorter wavelength than most visible colours of light, such as green and red. The inventors have found that blue light can cause an increase in growth rate, maximum biomass yield, pyruvate and ethanol production in photosynthetic microorganisms. This result was surprising as blue light has long been known to decrease the photosynthetic efficiency of cyanobacteria via photo bleaching and has been shown to result in cell death at high intensities (Hirosawa, 1984; Luimstra et al, 2018). Furthermore, studies have described that cyanobacteria use blue light less efficiently for photosynthesis than most phototrophs (Luimstra et al, 2018). However, the use of blue light in methods according to the present disclosure has now been shown to lead to production of >10-20 fold more ethanol than previously reported in the model organism Synechocystis, via upregulation of carbon flux through the pyruvate pathway.

As a result, blue light is used in this disclosure to upregulate the synthesis of pyruvate and/or metabolites of pyruvate in a photosynthetic microorganism. Optionally, blue light is not used for the purpose of activating heterologously expressed enzymes, e.g. a photodecarboxylase such as Chlorella vulgaris fatty acid photodecarboxylase (CvFAP). Upregulating the synthesis of pyruvate and/or a metabolite of pyruvate in a photosynthetic microorganism enables the photosynthetic production of one, two, three or four carbon compounds, and also enables the production of more complex products including isoprene, prenol, isoprenol, linalool, geraniol, farnesene, bisabolene or β-caryophyllene.

Accordingly, in some embodiments the photosynthetic microorganism culture is irradiated (illuminated) with blue light. Whereas ambient or background light is usually white light (being a mixture of visible light wavelengths across the approximate range of 380 nm and 760 nm) in some methods according to the present disclosure the light provided to (shone upon) the culture is ‘blue light’, defined herein as light that is biased in its wavelength composition to have a greater proportion of light having a wavelength in the range 380 to 500 nm (or one of the sub-ranges described below) than is usually present in white light, other colours of visible light or in ambient or background light. In this specification irradiation with blue light is to be understood in this context.

Reference to irradiation or illumination in this specification is preferably to be construed as excluding any background or ambient light that may be present in the container, vessel, room etc in which the culture is being undertaken. That is, it is to be understood in the context of the light that is being actively applied to the culture in order to stimulate photosynthesis. Such active application of light will typically be provided by a light source. The light source may be a blue light source, producing blue light as defined above.

The light source used to irradiate (illuminate) the culture will be any light source which is capable of providing blue light, as defined herein. Such a light source is herein referred to as a ‘blue light source’. As such, methods according to the present disclosure may involve irradiating a culture using a blue light source. Suitable blue light sources may include light-emitting diodes (LEDs), fluorescent light bulbs, or incandescent light bulbs capable of providing blue light, as defined herein.

There are many potential commercial light sources that are suitable for the culture of photosynthetic microorganisms.

One option is a fluorescent bulb, such as a Fluorescent Tube Lamp. Fluorescent Tube Lamps are available with different power capabilities, for example 20 watts, 40 watts, 65 watts, 75 watts and 100 watts. Light sources with different power outputs may be suitable as a light source for photosynthesis, but the difference in power may affect photosynthetic efficiency.

Fluorescent lamps can emit across a wide spectrum of wavelengths (300 nm and 800 nm). A commercial example of a fluorescent tube lamp is the Philips™ TL-X XL 40 W 33-640 4 Ft T12 TLX4033, which has wattage of 40 watts, a voltage of 103 V, and emits cool white light with a broad wavelength range (between 300 and 800 nm).

Fluorescent lamps with more specific wavelengths can also be utilised as a light source for photosynthesis. A commercial example of a fluorescent tube lamp which emits blue light is the Sylvania™ T8 Coloured Fluorescent Tube 58 W Blue 0002571, which has a wattage of 58 watts, a voltage of 240 v, and emits blue light.

LED lights may also be used as a light source for photosynthesis and are capable of producing light at a wide range of wavelengths.

One example of a commercial white light LED which could be used as a light source for photosynthesis is the Philips™ CorePro LEDbulb E27 A60 5.5 W 830, which has a wattage of 5.5 watts, a voltage of 220-240 v, and emits white light.

One example of a commercial blue light LED which could be used as a light source for photosynthesis is the Osram™ Parathom Classic Color E27 2 W|Blue, which has a wattage of 2 watts, a voltage of 220-240 v, and emits blue light.

In some embodiments the blue light source may produce light of a narrow range of wavelengths, e.g. of one of the wavelength ranges described below.

As described above, blue light has a wavelength between 380 nm and 500 nm. As such, the photosynthetic microorganism culture may be irradiated with light having a wavelength, and optionally also a spectral peak, between 380 nm and 500 nm. In some embodiments the photosynthetic microorganism culture may be irradiated with light having a wavelength, and optionally also a spectral peak, between one of 390 nm and 500 nm, 390 nm and 490 nm, 390 nm and 480 nm, 390 nm and 470 nm, 400 nm and 500 nm, 400 nm and 490 nm, 400 nm and 480 nm, 400 nm and 470 nm, 410 nm and 500 nm, 410 nm and 490 nm, 410 nm and 480 nm, 410 nm and 470 nm, 420 nm and 500 nm, 420 nm and 490 nm, 420 nm and 480 nm, 420 nm and 470 nm, 430 nm and 500 nm, 430 nm and 490 nm, 430 nm and 480 nm, 430 nm and 470 nm, 440 nm and 500 nm, 440 nm and 490 nm, 440 nm and 480 nm, 440 nm and 470 nm, 450 nm and 500 nm, 450 nm and 490 nm, 450 nm and 480 nm, 450 nm and 470 nm.

In some embodiments, blue light has a wavelength, and optionally also a spectral peak, between one of 430 nm and 450 nm, 430 nm and 440 nm, 440 nm and 460 nm, 440 nm and 450 nm, 450 nm and 470 nm, 450 nm and 460 nm, 460 nm and 480 nm, or 460 nm and 470 nm.

In some embodiments, the blue light has a spectral peak at one of about 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480 nm.

In some embodiments, the blue light has a spectral peak at one of about 430-433, 432-435, 434-437, 436-439, 438-441, 440-443, 442-445, 444-447, 446-449, 448-451, 450-453, 452-455, 454-457, 456-459, 458-461, 460-463, 462-465, 464-467, 466-469, 468-471, 470-473, 472-475, 474-477, 476-479, 480-483 nm.

In some embodiments, the blue light has a wavelength of less than one of 500, 495, 490, 485, or 480 nm, and optionally greater than 380 or 390 nm.

As described above, irradiation with blue light preferably refers to irradiation with light having a wavelength composition comprising a greater proportion of blue light than is usually present in white light, ambient or background light. This proportion may be one of 10%, 20%, 30%, 40%, 50%, 60%, 70%, 80%, or 90%, or 95% or more. Preferably, the proportion is more than one of 30% (e.g. 30%, 31%, 32%, 33%, 34% or more), 35% (e.g. 35%, 36%, 37%, 38%, 39% or more), 40% (e.g. 40%, 41%, 42%, 43%, 44% or more), 45% (e.g. 45%, 46%, 47%, 48%, 49%) 50% (e.g. 50%, 51%, 52%, 53%, 54%, 55%, or more).

As such, the proportion of the total visible light actively shone upon (e.g. emitted by the blue light source) or absorbed by the culture that is blue light is preferably greater than one of 10%, 20%, 30%, 40%, 50%, 60%, 70%, 80%, or 90%, or 95%. Accordingly, less than one of 5%, 10%, 20%, 30%, 40%, 50%, 60%, 70%, 80%, or 90% (respectively, considering the proportion of blue light shone on the culture) of the total visible light shone upon (e.g. emitted by the blue light source) or absorbed by the culture is light having a wavelength between 501 nm and 800 nm.

The wavelength of light can be observed through its light spectrum. In some cases the frequency spectrum may include a distinct peak. A peak is the wavelength where the spectrum reaches its highest intensity. The light that is actively shone upon the culture may be have a single peak of light or may have multiple peaks.

A single peak of blue light may have a peak of light within the spectrum of wavelength between 380 nm and 500 nm. The single peak of blue light may have a peak of light within the spectrum of wavelengths between one of 390 nm and 500 nm, 390 nm and 490 nm, 390 nm and 480 nm, 390 nm and 470 nm, 400 nm and 500 nm, 400 nm and 490 nm, 400 nm and 480 nm, 400 nm and 470 nm, 410 nm and 500 nm, 410 nm and 490 nm, 410 nm and 480 nm, 410 nm and 470 nm, 420 nm and 500 nm, 420 nm and 490 nm, 420 nm and 480 nm, 420 nm and 470 nm, 430 nm and 500 nm, 430 nm and 490 nm, 430 nm and 480 nm, 430 nm and 470 nm, 440 nm and 500 nm, 440 nm and 490 nm, 440 nm and 480 nm, 440 nm and 470 nm, 450 nm and 500 nm, 450 nm and 490 nm, 450 nm and 480 nm, 450 nm and 470 nm.

Alternatively, the light that is actively shone upon the culture may have multiple peaks. The peaks may be within the spectrum of wavelength between 380 nm and 500 nm. The peaks of light may be within the spectrum of wavelengths between one of 390 nm and 500 nm, 390 nm and 490 nm, 390 nm and 480 nm, 390 nm and 470 nm, 400 nm and 500 nm, 400 nm and 490 nm, 400 nm and 480 nm, 400 nm and 470 nm, 410 nm and 500 nm, 410 nm and 490 nm, 410 nm and 480 nm, 410 nm and 470 nm, 420 nm and 500 nm, 420 nm and 490 nm, 420 nm and 480 nm, 420 nm and 470 nm, 430 nm and 500 nm, 430 nm and 490 nm, 430 nm and 480 nm, 430 nm and 470 nm, 440 nm and 500 nm, 440 nm and 490 nm, 440 nm and 480 nm, 440 nm and 470 nm, 450 nm and 500 nm, 450 nm and 490 nm, 450 nm and 480 nm, 450 nm and 470 nm.

In some embodiments, the light that is actively shone upon the culture has no peaks of red light, orange light, yellow light and/or green light. In a preferred embodiment the light that is actively shone upon the culture does not have a peak of red light, or contains no red light. Accordingly, the light that is actively shone upon the culture may have no peaks outside the spectrum of wavelengths between one of 380 nm and 500 nm, 390 nm and 500 nm, 390 nm and 490 nm, 390 nm and 480 nm, 390 nm and 470 nm, 400 nm and 500 nm, 400 nm and 490 nm, 400 nm and 480 nm, 400 nm and 470 nm, 410 nm and 500 nm, 410 nm and 490 nm, 410 nm and 480 nm, 410 nm and 470 nm, 420 nm and 500 nm, 420 nm and 490 nm, 420 nm and 480 nm, 420 nm and 470 nm, 430 nm and 500 nm, 430 nm and 490 nm, 430 nm and 480 nm, 430 nm and 470 nm, 440 nm and 500 nm, 440 nm and 490 nm, 440 nm and 480 nm, 440 nm and 470 nm, 450 nm and 500 nm, 450 nm and 490 nm, 450 nm and 480 nm, 450 nm and 470 nm.

As described above, irradiation with blue light preferably refers to irradiation with light having a wavelength composition comprising a greater proportion of blue light than is usually present in white light, ambient or background light. Accordingly, the light that is actively shone upon the culture has less red light, orange light, yellow light and/or green light than that present in white light. In some embodiments, less than one of 5%, 10%, 20%, 30%, 40%, 50%, 60%, 70%, 80%, or 90% (respectively, considering the proportion of blue light shone on the culture) of the total visible light shone upon (e.g. emitted by the blue light source) or absorbed by the culture is red light, orange light, yellow light and/or green light. In most preferred embodiments, less than one of 5%, 10%, 20%, 30%, 40%, 50%, 60%, 70%, 80%, or 90% (respectively, considering the proportion of blue light shone on the culture) of the total visible light shone upon (e.g. emitted by the blue light source) or absorbed by the culture is red light.

Irradiation with blue light may also be of blue light having a certain photon dose (photon flux or photon flux density; being the number of photons of light of desired wavelength passing through a unit area in a unit time), which may be measured in μE or the equivalent SI unit of μmol m⁻²s⁻¹, optionally measured at a distance of 1 cm or 10 cm. Accordingly, a blue light source may emit blue light of a defined photon dose. Preferably photon dose in this specification refers to the photon flux or photon flux density which is applied to the culture.

In some embodiments, the photon dose may be greater than one of 10, 20, 30, 40, 50, 60, 70, 80, 90, 100, 150, 200, 250, 300, 350, 400, 450, 500, 550, 600, 650, 700, or 750 μmol m⁻²s⁻¹.

In some embodiments, light having a wavelength between 380 nm and 500 nm is provided at a photon dose of less than one of 3000, 2000, 1000, 950, 900, 850, 800, 750, 700, 650, 600, 550, or 500 μmol m⁻² s⁻¹.

In some embodiments, the culture is irradiated with blue light at a photon dose of 10 to 1000 μmol m⁻²s⁻¹, or more.

In some embodiments, the photon dose may be one of between 10 to 1000, 20 to 1000, 30 to 1000, 40 to 1000, 50 to 1000, 60 to 1000, 70 to 1000, 80 to 1000, 90 to 1000, 100 to 1000, 110 to 1000, 120 to 1000, 130 to 1000, 140 to 1000, 150 to 1000, 200 to 1000, 250 to 1000, 300, to 1000, 400 to 1000, 500 to 1000, 600 to 1000, 700 to 1000, 800 to 1000, or 900 to 1000 μmol m⁻²s⁻¹.

In some embodiments, the photon dose may be one of between 10 to 2000, 20 to 2000, 30 to 2000, 40 to 2000, 50 to 2000, 60 to 2000, 70 to 2000, 80 to 2000, 90 to 2000, 100 to 2000, 110 to 2000, 120 to 2000, 130 to 2000, 140 to 2000, 150 to 2000, 200 to 2000, 250 to 2000, 300 to 2000, 400 to 2000, 500 to 2000, 600 to 2000, 700 to 2000, 800 to 2000, or 900 to 2000 μmol m⁻²s⁻¹.

In some embodiments, the photon dose may be one of between 10 to 3000, 20 to 3000, 30 to 3000, 40 to 3000, 50 to 3000, 60 to 3000, 70 to 3000, 80 to 3000, 90 to 3000, 100 to 3000, 110 to 3000, 120 to 3000, 130 to 3000, 140 to 3000, 150 to 3000, 200 to 3000, 250 to 3000, 300, to 3000, 400 to 3000, 500 to 3000, 600 to 3000, 700 to 3000, 800 to 3000, or 900 to 3000 μmol m⁻²s⁻¹.

In some embodiments, the photon dose may be one of between 10 to 800, 20 to 800, 30 to 800, 40 to 800, 50 to 800, 60 to 800, 70 to 800, 80 to 800, 90 to 800, 100 to 800, 110 to 800, 120 to 800, 130 to 800, 140 to 800, 150 to 800, 200 to 800, 250 to 800, 300, to 800, 400 to 800, 500 to 800, 600 to 800, or 700 to 800 μmol m⁻²s⁻¹.

In some embodiments, the photon dose may be one of between 10 to 700, 20 to 700, 30 to 700, 40 to 700, 50 to 700, 60 to 700, 70 to 700, 80 to 700, 90 to 700, 100 to 700, 110 to 700, 120 to 700, 130 to 700, 140 to 700, 150 to 700, 200 to 700, 250 to 700, 300, to 700, 400 to 700, 500 to 700, or 600 to 700 μmol m⁻²s⁻¹.

In some embodiments, the photon dose may be one of between 10 to 800, 20 to 800, 30 to 800, 40 to 600, 50 to 600, 60 to 600, 70 to 600, 80 to 600, 90 to 600, 100 to 600, 110 to 700, 120 to 700, 130 to 700, 140 to 600, 150 to 600, 200 to 600, 250 to 600, 300 to 600, 400 to 600, or 500 to 600 μmol m⁻²s⁻¹.

In some embodiments, the photon dose may be one of between 10 to 500, 20 to 500, 30 to 500, 40 to 500, 50 to 500, 60 to 500, 70 to 500, 80 to 500, 90 to 500, 100 to 500, 110 to 500, 120 to 500, 130 to 500, 140 to 500, 150 to 500, 200 to 500, 250 to 500, 300 to 500, or 400 to 500 μmol m⁻²s⁻¹.

In some embodiments, the photon dose may be one of between 10 to 250, 20 to 250, 30 to 250, 40 to 250, 50 to 250, 60 to 250, 70 to 250, 80 to 250, 90 to 250, 100 to 250, 110 to 250, 120 to 250, 130 to 250, 140 to 250, 150 to 250, 160 to 250, 170 to 250, 180 to 250, 190 to 250, 200 to 250, 210 to 250, 220 to 250, 230 to 250, or 240 to 250 μmol m⁻²s⁻¹.

In some embodiments, the photon dose may be one of between 10 to 150, 20 to 150, 30 to 150, 40 to 150, 50 to 150, 60 to 150, 70 to 150, 80 to 150, 90 to 150, 100 to 150, 110 to 150, 120 to 150, 130 to 150, or 140 to 150 μmol m⁻²s⁻¹.

The photon dose may vary depending on the size of the culture. For example, a small culture will require irradiation with blue light at a smaller photon dose than a large culture. Small cultures have reduced shading within the culture and a lower photon dose is required to penetrate to the centre of the culture when compared to a larger culture.

In some embodiments, the culture is irradiated with blue light for an amount of time sufficient to upregulate synthesis of or production of a product of interest in/by the photosynthetic microorganism. The period of time selected may reflect the size of the culture, type of culture (e.g. batch or continuous), culture conditions, availability of nutrients, type of microorganism and/or product of interest.

In the case of a continuous culture the period of time may correspond to the period of time between collection of batches culture media/fluid from which the product of interest is extracted or isolated.

In some embodiments the period of time may be one of at least 5, 10, 15, 30, 45, or 60 minutes. In some embodiments the period of time may be one of at least 1, 2, 3, 4, 5, 6, 7, 8, 9, 10, 11, 12, 13, 14, 15, 16, 17, 18, 19, 20, 21, 22, 23, or 24 hours. In some embodiments the period of time may be one of at least 1, 2, 3, 4, 5, 6, or 7 days. In some embodiments the period of time may be one of at least 1, 2, 3, or 4 weeks.

In some embodiments the period of time may be 1 day or less, e.g. one of between 1 to 24, 2 to 24, 3 to 24, 4 to 24, 5 to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, or 23 to 24 hours.

In other embodiments the period of time may be more than one day, e.g. one of between 1 to 2, 1 to 3, 1 to 4, 1 to 5, 1 to 6, 1 to 7, 2 to 7, 3 to 7, 4 to 7, 5 to 7, or 6 to 7 days.

In some embodiments, the photosynthetic microorganisms may be grown under light-dark cycling conditions. In some embodiments, the photosynthetic microorganisms may be grown in 12 hour light and 12 hour dark cycles (12 h/12 h). In other embodiments, the photosynthetic microorganisms may be grown in 13 hour light and 11 hour dark cycles (13 h/11 h), 14 hour light and 10 hour dark cycles (14 h/10 h), 15 hour light and 9 hour dark cycles (15 h/9 h), 16 hour light and 8 hour dark cycles (16 h/8 h), 17 hour light and 7 hour dark cycles (17 h/7 h), 18 hour light and 6 hour dark cycles (18 h/6 h), 19 hour light and 5 hour dark cycles (19 h/5 h), 20 hour light and 4 hour dark cycles (20 h/4 h), 21 hour light and 3 hour dark cycles (21 h/3 h), 22 hour light and 2 hour dark cycles (22 h/2 h), or 23 hour light and 1 hour dark cycles (13 h/11 h).

During the light cycle, light of specific wavelengths can be applied to the culture, and during the dark cycle, no light is applied to the culture.

In some embodiments, the culture is irradiated with blue light for a period of time which is greater than a single light cycle. For example, in some embodiments, the culture is irradiated with blue light for 24 hours, and this blue light is provided over two 12 hour light cycles (i.e. 12 hours of blue light is provided before 12 hours of dark, which is then followed by a further 12 hours of blue light). In some embodiments, an initial high dose of blue light is applied, followed by irradiance with a lower dose of blue light.

In some embodiments, the culture is irradiated with blue light at a high photon dose for a period of time, before reducing the photon dose to a lower dose for a period of time.

The high dose may be in the general range 400 to 1000 μmol m⁻²s⁻¹.

In some embodiments the high dose may be between one of 600 to 1000, 700 to 1000, 800 to 1000, 900 to 1000 μmol m⁻²s⁻¹, or more.

In some embodiments the high dose may be between one of 600 to 2000, 700 to 2000, 800 to 2000, 900 to 2000 μmol m⁻²s⁻¹.

In some embodiments the high dose may be between one of 600 to 3000, 700 to 3000, 800 to 3000, 900 to 3000 μmol m⁻²s⁻¹.

In some embodiments the high dose may be between one of 500 to 800, 600 to 800, 700 to 800 μmol m⁻² s⁻¹.

In some embodiments the high dose may be between one of 400 to 700, 500 to 700, or 600 to 700 μmol m⁻²s⁻¹.

The high dose may be one of about 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μmol m⁻²s⁻¹.

The lower dose may be in the general range 10 to 600 μmol m⁻²s⁻¹.

In some embodiments the lower dose may be between one of 10 to 600, 20 to 600, 30 to 600, 40 to 600, 50 to 600, 60 to 600, 70 to 600, 80 to 600, 90 to 600, 100 to 600, 110 to 600, 120 to 600, 130 to 600, 140 to 600, 150 to 600, 200 to 600, 250 to 600, 300 to 600, 350 to 600, 400 to 600, 450 to 600, 500 to 600, 550 to 600 μmol m⁻²s⁻¹.

In some embodiments the lower dose may be between one of 10 to 500, 20 to 500, 30 to 500, 40 to 500, 50 to 500, 60 to 500, 70 to 500, 80 to 500, 90 to 500, 100 to 500, 110 to 500, 120 to 500, 130 to 500, 140 to 500, 150 to 500, 200 to 500, 250 to 500, 300 to 500, 350 to 500, 400 to 500, 450 to 500 μmol m⁻²s⁻¹.

The lower dose may be one of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 μmol m⁻²s⁻¹.

In some embodiments, the culture is irradiated with blue light at a photon dose between 700 μmol m⁻²s⁻¹ and 1000 μM, before reducing the photon dose to irradiate blue light at a photon dose between 10 μmol m⁻²s⁻¹ and 700 μM.

In some embodiments, the culture is irradiated with blue light at a photon dose between 750 μmol m⁻²s⁻¹ and 1000 μM, 800 μmol m⁻²s⁻¹ and 1000 μM, 850 μmol m⁻²s⁻¹ and 1000 μM, 750 μmol m⁻²s⁻¹ and 950 μM, 800 μmol m⁻²s⁻¹ and 950 μM, 750 μmol m⁻²s⁻¹ and 900 μM, 800 μmol m⁻²s⁻¹ and 900 μM, 850 μmol m⁻²s⁻¹ and 900 μM, 750 μmol m⁻²s⁻¹ and 900 μM, 750 μmol m⁻²s⁻¹ and 850 μM, 800 μmol m⁻²s⁻¹ and 850 μM, before reducing the photon dose to irradiate blue light at a photon dose between 10 μmol m⁻²s⁻¹ and 700 μM, 50 μmol m⁻²s⁻¹ and 700 μmol m⁻²s⁻¹, 100 μmol m⁻²s⁻¹ and 700 μmol m⁻²s⁻¹, 100 μmol m⁻²s⁻¹ and 600 μmol m⁻²s⁻¹, 150 μmol m⁻²s⁻¹ and 600 μmol m⁻²s⁻¹, 200 μmol m⁻²s⁻¹ and 600 μmol m⁻²s⁻¹, 300 μmol m⁻²s⁻¹ and 600 μmol m⁻²s⁻¹, 400 μmol m⁻²s⁻¹ and 600 μmol m⁻²s⁻¹, or 450 μmol m⁻²s⁻¹ and 600 μmol m⁻² s⁻¹.

The period of time for which the culture is irradiated with blue light at a high photon dose may be chosen to be sufficient to promote carbon flux through the pyruvate pathway, e.g, to upregulate pyruvate synthesis or a synthesis of a metabolite of pyruvate. As prolonged exposure to high dose blue light may lead to death of the microorganisms the period of time may be short, e.g. less than 6 hours. This period of time may be considered a ‘stimulation phase’ in which high dose blue light is used to stimulate an increase in carbon flux through the pyruvate pathway. As such, the period of time may be between 1 minute and about 6 hours. In some embodiments the period of time may be one of between 1 and 3, 1 and 5, 1 and 10, 1 and 15 minutes. In some embodiments the period of time may be one of between 2 and 5, 2 and 10, 2 and 15 minutes. In some embodiments the period of time may be one of between 5 and 10, 5 and 15, 5 and 30, 5 and 60 minutes. In some embodiments the period of time may be one of between 10 and 20, 10 and 30, 10 and 60 minutes. In some embodiments the period of time may be one of between 1 and 2, 1 and 3, 2 and 3, 2 and 4, 3 and 4, 3 and 5, 4 and 5, 4 and 6 hours.

The period of time for which the culture is irradiated with blue light at a lower photon dose may be chosen to be sufficient to maintain carbon flux through the pyruvate pathway whilst allowing the culture to be maintained without substantial cell death. This period of time may be considered a ‘maintenance phase’ in which lower dose blue light is used to maintain carbon flux through the pyruvate pathway without incurring substantial cell death. The maintenance phase will typically be longer than the stimulation phase. As such, the period of time may be more than 1 hour and up to several weeks, or more. In some embodiments the period of time may be one of between 1 to 24, 2 to 24, 3 to 24, 4 to 24, 5 to 24, 6 to 24, 7 to 24, 8 to 24, 9 to 24, 10 to 24, 11 to 24, 12 to 24, 13, to 24, 14 to 24, 15 to 24, 16 to 24, 17 to 24, 18 to 24, 19 to 24, 20 to 24, 21 to 24, 22 to 24 or 23 to 24 hours. In other embodiments the period of time may be more than one day, e.g. one of between 1 to 2, 1 to 3, 1 to 4, 1 to 5, 1 to 6, 1 to 7, 2 to 7, 3 to 7, 4 to 7, 5 to 7, or 6 to 7 days. In other embodiments the period of time may be one week or more, e.g. one of between 1 to 2, 1 to 3, 1 to 4 weeks.

The inventors have shown that whilst culture under blue light conditions increases the metabolic flux through pyruvate, prolonged irradiation with blue light can lead to death of the microorganisms. As such, an initial stage of culture intended to promote growth of the microorganisms to a desirable cell density may be performed prior to irradiation with blue light. This initial stage may be conducted under normal light (e.g. white light or non-blue light) conditions. As such, the photosynthetic microorganism culture may be irradiated with white light prior to being cultured under irradiation with blue light.

Cell density of a photosynthetic microorganism culture is simple to measure using a spectrophotometer. The optical density (OD) can be measured by determining the degree to which the culture retards transmitted rays of light. Therefore, the higher the OD, the more dense the culture. Different wavelengths can be used to determine the OD of cultures from different organisms. Suitable wavelengths to determine the OD of photosynthetic microorganisms are 680 nm (OD₆₈₀) or 720 nm (OD₇₂₀).

In some embodiments, the photosynthetic microorganism culture is grown under normal light (e.g. white light or non-blue light) conditions until the photosynthetic microorganism is grown to a high cell density, determined by an OD₆₈₀ measurement of over 0.5, before irradiation is changed to blue light for a selected period of time.

In some embodiments, the photosynthetic microorganism culture is grown under normal light (e.g. white light or non-blue light) conditions until the photosynthetic microorganism is grown to a high cell density, determined by an OD₆₈₀ measurement of over 0.6, 0.7, 0.8, 0.9, 1.0, or 1.1, before irradiation is changed to blue light for a selected period of time.

Ethanol Production

In some embodiments, the culture is irradiated with blue light, for a period of time sufficient to produce ethanol at a concentration of at least 2% (w/v) in the culture.

In other embodiments, the culture is irradiated with blue light, for a period of time sufficient to produce ethanol at a concentration of at least 3% (w/v), 4% (w/v), 5% (w/v), 6% (w/v), 7% (w/v), 8% (w/v), 9% (w/v), or 10% (w/v) in the culture.

In some embodiments, the culture is irradiated with blue light, at a dose sufficient to produce ethanol at a concentration of at least 2% (w/v) in the culture.

In other embodiments, the culture is irradiated with blue light, at a dose sufficient to produce ethanol at a concentration of at least 3% (w/v), 4% (w/v), 5% (w/v), 6% (w/v), 7% (w/v), 8% (w/v), 9% (w/v), or 10% (w/v) in the culture.

The concentration of ethanol in a culture in terms of weight by volume (w/v) is simple to calculate through standard methods known in the art. w/v (%)=mass of solute (g)÷volume of solution (mL)×100.

Ethanol productivity can be calculated simply based on the amount of ethanol produced within a defined volume over a certain amount of time. Ethanol productivity can be calculated in terms of the grams of ethanol produced per litre per day (g/L/day), and can also be calculated in terms of the grams of ethanol produced per litre per hour (g/L/h). The current record titre of photosynthetic cyanobacterial ethanol production is 5.5 g/L after 26 days, or 2 g/L/day (Lehtinen et al., 2018). 2 g/L/day equates to 0.08 g/L/h.

In some embodiments, the culture is irradiated with blue light, for a period of time sufficient to produce ethanol at over 0.1 g/L/h.

In other embodiments, the culture is irradiated with blue light, at a dose sufficient to produce ethanol at over 0.2 g/L/h, 0.3 g/L/h, 0.4 g/L/h, 0.5 g/L/h, 0.6 g/L/h, 0.7 g/L/h, 0.8 g/L/h, 0.9 g/L/h, 1.0 g/L/h, 1.1 g/L/h, 1.2 g/L/h, 1.3 g/L/h, 1.4 g/L/h, 1.5 g/L/h, 1.6 g/L/h, 1.7 g/L/h, 1.8 g/L/h, 1.9 g/L/h, 2.0 g/L/h, 2.5 g/L/h, 3.0 g/L/h, 3.5 g/L/h, 4.0 g/L/h, 4.5 g/L/h, 5.0 g/L/h, or 5.5 g/L/h.

In some embodiments, the culture is irradiated with blue light, for a period of time sufficient to produce ethanol at over 0.1 g/L/h.

In other embodiments, the culture is irradiated with blue light, at a dose sufficient to produce ethanol at over 0.2 g/L/h, 0.3 g/L/h, 0.4 g/L/h, 0.5 g/L/h, 0.6 g/L/h, 0.7 g/L/h, 0.8 g/L/h, 0.9 g/L/h, 1.0 g/L/h, 1.1 g/L/h, 1.2 g/L/h, 1.3 g/L/h, 1.4 g/L/h, 1.5 g/L/h, 1.6 g/L/h, 1.7 g/L/h, 1.8 g/L/h, 1.9 g/L/h, 2.0 g/L/h, 2.5 g/L/h, 3.0 g/L/h, 3.5 g/L/h, 4.0 g/L/h, 4.5 g/L/h, 5.0 g/L/h, or 5.5 g/L/h.

In some embodiments, the culture is irradiated with blue light, at a dose sufficient to produce ethanol at a concentration of at least 2% (w/v) in the culture.

In other embodiments, the culture is irradiated with blue light, at a dose sufficient to produce ethanol at a concentration of at least 3% (w/v), 4% (w/v), 5% (w/v), 6% (w/v), 7% (w/v), 8% (w/v), 9% (w/v), or 10% (w/v) in the culture.

Photosynthesis

Photosynthesis is a process which can occur in plants and microorganisms, in which light energy is converted into chemical energy. Energy from light is absorbed by the reaction centres, which contain pigments such as chlorophyll, carotenoids and phycobilins,

The process fixes carbon from the environment and produces carbon compounds which are either used or stored by the organism. Requirements for photosynthesis to occur in photosynthetic microorganisms are the supply of a carbon source, a light source and water.

The simple overall equation for photosynthesis is: 6CO₂+6H₂O→C₆H₁₂O₆+6O₂

However, photosynthesis is a complex multi-reaction process, involving light dependent and light independent reactions.

In the light dependent reactions, photons of light are absorbed by the reaction centres to synthesise two molecules needed for the next stage of photosynthesis: the energy storage molecule ATP and the reduced electron carrier NADPH. The net-reaction of all light-dependent reactions in oxygenic photosynthesis is: 2H₂O+2NADP⁺+3ADP+3Pi→O₂+2NADPH+3ATP

The light independent reactions are also known as the Calvin cycle. It is at this stage that carbon is fixed. In the Calvin cycle, carbon atoms from CO₂ are fixed (incorporated into organic molecules) and used to build three-carbon sugars. This process is fuelled by, and dependent on, ATP and NADPH from the light reactions.

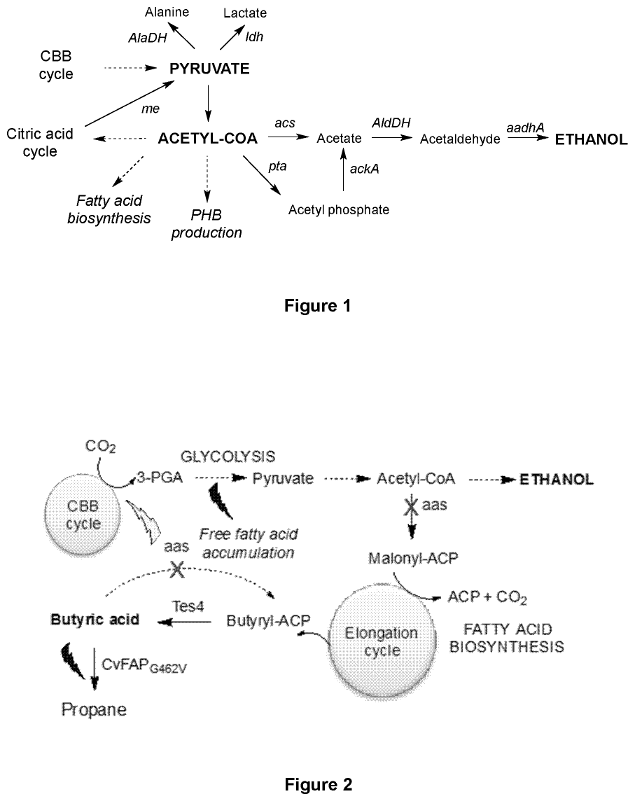

One product from the Calvin cycle is 3-phosphoglycerate (3-PGA). 3-PGA is a three carbon compound and is the product of the scission of an unstable 6-carbon intermediate formed upon CO₂ fixation. As shown in FIG. 2, 3-PGA can then be converted to pyruvate. The conversion of 3-PGA to pyruvate is performed via three steps. Firstly, 3-PGA is converted to 2-phosphoglycerate (2-PGA) through the action of phosphoglycerate mutase. Secondly, 2-PGA is converted to phosphoenol pyruvic acid through the action of enolase. Thirdly, phosphoenol pyruvic acid is converted to pyruvate through the action of pyruvate kinase.

Pyruvate and Metabolites of Pyruvate

Pyruvate, or pyruvic acid, is a three carbon compound with the chemical formula C₃H₄O₃.

In some embodiments, the method upregulates the synthesis of pyruvate in a photosynthetic microorganism.

Pyruvate is an intermediate in many biochemical reactions which have applications in biotechnology. Specifically, in the case of fatty acid, polyhydroxybutyrate (PHB) and ethanol biosynthesis, pyruvate is a key intermediate in their synthesis. Pyruvate is also the starting point of many complex anabolic pathways such as the MEP pathway that results in chlorophyll and terpenoid biosynthesis (Pattanaik and Lindberg, 2015). Therefore it is of interest to develop methods which upregulate the synthesis of pyruvate.

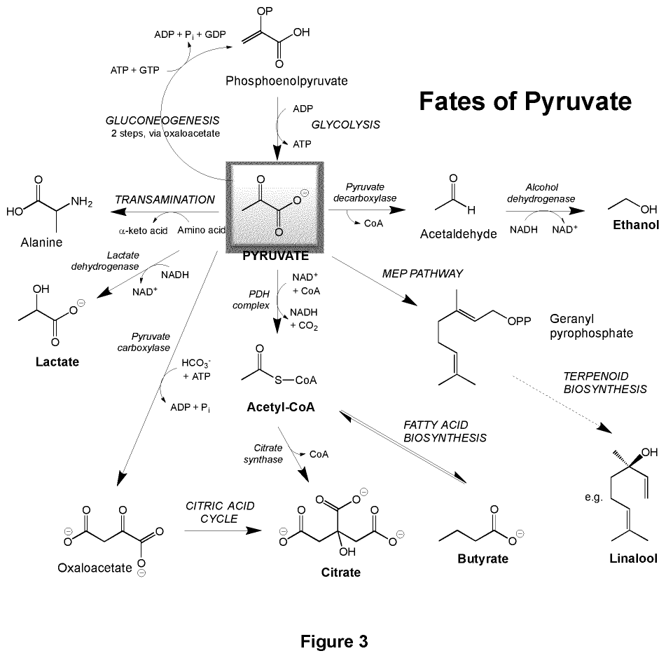

As an intermediate in a number of different biochemical reactions, there are a number of potential metabolites of pyruvate, some of which are shown in FIG. 3.

The flux of pyruvate in Synechocystis, relative to 100% CO₂ uptake, is 9.5% through pyruvate kinase and 5.3% through malic enzyme (Young et al., 2011). Therefore, approximately 5 to 10% of fixed carbon is destined to end up as pyruvate and its metabolites (or derivatives) in standard conditions.

In some embodiments, the method upregulates the synthesis of metabolites of pyruvate in a photosynthetic microorganism.

In this application, a metabolites of pyruvate is to be defined as any compound which is derived from pyruvate in a metabolic pathway present in a photosynthetic microorganism. A metabolite of pyruvate can be directly derived from pyruvate (i.e. from a single reaction), or can be indirectly derived from pyruvate (i.e. from more than one reaction).

In some embodiments, the method upregulates the synthesis of one or more metabolites of pyruvate, such as one or more of ethanol, acetyl-CoA, alanine, butanol, propane, acetate, or lactate

In some embodiments, the method may be part of a method for the production of more complex products, via the native anabolic pathways from pyruvate, such as the MEP pathway. Examples of such complex products include isoprene, prenol, isoprenol, linalool, geraniol, farnesene, bisabolene or β-caryophyllene. In some embodiments, the metabolite of pyruvate is not butyric acid or propane.

In some embodiments, the method upregulates the synthesis of one, two, three or four carbon containing compounds such as pyruvate, ethanol, acetyl-CoA, alanine, butanol, propane, acetate, or lactate.

In some embodiments, the method is for the photosynthetic production of a one, two, three or four carbon compound, which may be isolated from the photosynthetic microorganism or their culture. For example, ethanol may be isolated by distillation or pervaporation.

One, two, three or four carbon containing compounds are any compounds which contain between one and four carbons. For example, methane is a one carbon compound, ethanol is a two carbon compound, propane is a three carbon compound and butanol is a four carbon compound.

Examples of one, two, three or four carbon containing compounds which may be synthesised, produced or isolated are methane, ethanol, acetyl-CoA, alanine, butanol, propane, acetate, butyrate, ethane, ethene, propene, butene or lactate. The carbon flux through pyruvate and acetyl-CoA, including native ethanol biosynthesis, is shown in FIG. 1.

In some embodiments, the one, two, three or four carbon containing compound is not butyric acid or propane. One metabolite of pyruvate of particular interest is ethanol. Ethanol is a two carbon compound with the chemical formula C₂H₆O, which may be produced in photosynthetic microorganisms from pyruvate as shown in FIG. 1. It can be seen in FIG. 1 that pyruvate can be converted to acetyl CoA through pyruvate dehydrogenase activity, which can then be converted to acetate through acetate synthase activity, before conversion to acetaldehyde through aldehyde dehydrogenase activity and then to ethanol through acetaldehyde dehydrogenase activity. In some embodiments, the method upregulates the synthesis of ethanol in a photosynthetic microorganism.

In some embodiments, the method upregulates the synthesis of alanine in a photosynthetic microorganism. As shown in FIGS. 1 and 3, pyruvate can be converted to alanine through the transamination activity of alanine dehydrogenase.

In some embodiments, the method upregulates the synthesis of propane in a photosynthetic microorganism. As shown in FIG. 2, propane is a metabolite of pyruvate. Pyruvate can be converted to acetyl CoA through pyruvate dehydrogenase activity, which can then be converted to malonyl-ACP through acyl-acyl carrier protein synthase activity, malonyl-ACP can be converted to butyryl-ACP through standard fatty acid biosynthesis pathways, butyryl-ACP can be converted to butyric acid through thioesterase activity and butyric acid can be converted to propane through decarboxylase activity (e.g. fatty acid decarboxylase or CvFAP).

In some embodiments, the method upregulates the synthesis of butanol in a photosynthetic microorganism. Pyruvate can be converted to acetyl-CoA through pyruvate dehydrogenase activity, which can then be converted to malonyl-ACP through acyl-acyl carrier protein synthase activity, malonyl-ACP can be converted to butyryl-ACP through standard fatty acid biosynthesis pathways, butyryl-ACP can be converted to butyric acid through thioesterase activity and butyric acid can be converted to butyraldehyde through carboxylic acid reductase activity, and butyraldehyde can be converted to butanol through aldehyde-alcohol dehydrogenase activity.

In some embodiments, the method upregulates the synthesis of acetate in a photosynthetic microorganism. As shown in FIG. 1, pyruvate can be converted to acetyl-CoA through pyruvate dehydrogenase activity, and acetyl-CoA can be which can then be converted to acetate through acetate synthase activity.

In some embodiments, the method upregulates the synthesis of butyrate in a photosynthetic microorganism. Butyrate is a metabolite of pyruvate. Pyruvate can be converted to acetyl-CoA through pyruvate dehydrogenase activity, which can then be converted to malonyl-ACP through acyl-acyl carrier protein synthase activity, malonyl-ACP can be converted to butyryl-ACP through standard fatty acid biosynthesis pathways and butyryl-ACP can be converted to butyrate through thioesterase activity.

In some embodiments, the method upregulates the synthesis of lactate in a photosynthetic microorganism. As shown in FIG. 1 and FIG. 3, pyruvate can be converted to lactate through the action of lactate dehydrogenase.

In some embodiments, more complex compounds with a higher carbon content (C5-C20) can be produced through the native anabolic pathways from pyruvate. One such native anabolic pathway is the methylerythritol 4-phosphate (MEP) pathway which leads to the formation of various isoprenoids. Native anabolic pathways could lead to the synthesis of compounds such as isoprene, prenol, isoprenol, linalool, geraniol, farnesene, bisabolene or β-caryophyllene from pyruvate.

In some embodiments, the method upregulates the synthesis of terpenoids. Terpenoids are synthesized from the isoprene building blocks dimethylallyl pyrophosphate (DMAPP) and isopentenyl pyrophosphate (IPP). Combination of DMAPP and IPP generates pyrophosphate substrates of varying carbon lengths, which are then utilized by terpene synthases to produce either monoterpenes (C10), sesquiterpenes (C15), diterpenes (C20), or others.

FIG. 3 shows, by way of example, that geranyl pyrophosphate (GPP) can be produced from pyruvate through the MEP pathway. Geranyl pyrophosphate (GPP), the substrate used by monoterpene synthases, is formed by coupling one molecule of DMAPP with IPP, while farnesyl pyrophosphate (FPP), the substrate for sesquiterpenes, is synthesized by coupling three individual isoprene precursors (Oldfield et al., 2012).

Biosynthesis of Carbon Compounds

Traditionally, the biosynthesis of ethanol is performed in yeast, through anaerobic fermentation of biomass to ethanol. However, this requires a large amount of feedstock material (˜200-500 g/L sugars), contributing to the significant costs of the process. This means that the biosynthesis of ethanol in yeast can be expensive and it has an impact on the environment.

The biosynthesis of carbon compounds, including ethanol, in photosynthetic microorganisms has the potential to be cheaper and better for the environment. Carbon can be fixed from the environment or industry as CO₂ and converted to useful products through photosynthesis and subsequent biochemical reactions.

At present, the biosynthesis of ethanol is not commercially viable in photosynthetic microorganisms. The present disclosure is designed to address this issue. The current record titre of photosynthetic cyanobacterial ethanol production is 5.5 g/L (0.55% w/v) after 26 days, or 2 g/L/day (Lehtinen et al., 2018).

Distillation-based ethanol recovery processes are required to make the recovery of ethanol commercially viable. An ethanol concentration of at least 10% (w/v) in the culture is usually sought to make distillation from culture economically viable. This is considerably higher than the 0.55% seen in the prior art.

Isolation of Carbon Compounds

In some embodiments, carbon compounds of interest, such as ethanol, are secreted into the media and can be isolated from the media.

In other embodiments, carbon compounds of interest are not secreted and need to be isolated from the photosynthetic microorganism fraction.

The first step for both of these methods is to separate the photosynthetic microorganism fraction from the liquid fraction. The liquid fraction is the liquid portion (e.g. culture media) which lies above a sediment formed by the solid photosynthetic microorganisms. The photosynthetic microorganism fraction is the solid sediment fraction which forms below the liquid fraction.

The photosynthetic microorganism fraction can be separated from the liquid fraction in a number of ways, including filtration, chromatography, evaporation, sedimentation, and centrifugation. Following this crude separation, the compounds of interest can be separated from their fraction.

Compounds that are secreted into the media from photosynthetic microorganisms and present in the liquid fraction can be separated in a number of ways, including distillation and pervaporation. For example, the separation of ethanol from the other components of the media liquid fraction is essentially the separation of ethanol from water with the addition of impurities.

Distillation is the process of separating the components or substances from a liquid mixture by using selective boiling and condensation. The process takes advantage of the fact that different compounds have different boiling points. There are a number of different types of distillation that could be used to separate carbon compounds from media liquid fraction: simple distillation, fractional distillation, vacuum distillation, and azeotropic distillation, to name a few.

Other compounds of interest which are not secreted, and need to be isolated from the photosynthetic microorganism fraction, require an additional processing step to isolate the compound. To make the compound available for separation, the cells may need to be lysed. Cells can be lysed through mechanical homogenization, ultrasonic homogenisation, pressure homogenisation, freeze-thaw treatment, heat treatment, osmotic lysis and chemical lysis. Following this, the compounds can be isolated through methods known in the art.

Remaining cell mass from the photosynthetic microorganism fraction can be harvested and used as either fertiliser or in animal feeds, or as feedstock for other biotechnological processes. These products are often high in nutrients, minerals, protein, oil, and/or carbohydrates, and have value as fertiliser or in animal feeds. The remaining cell mass can be either whole cells or the solid fraction of lysed cells.

In some embodiments, the photosynthetic microorganisms die after irradiation with blue light, and the culture is removed from the growth container for product isolation (culture removal), and replaced with a new culture of photosynthetic microorganisms (culture replacement).

In some embodiments, there is a cycling, continuous system of (1) irradiation with blue light, (2) culture removal, (3) culture replacement, and then the cycle starts again from step 1.

In some embodiments, there is a cycling, continuous system of (1) irradiation with white light, (2) irradiation with blue light, (3) culture removal, (4) culture replacement, and then the cycle starts again from step 1.

Modified Photosynthetic Microorganisms

In some embodiments, a method is provided for the production of pyruvate or a metabolite of pyruvate from a photosynthetic microorganism, the method comprising culturing the photosynthetic microorganism under conditions suitable for photosynthesis, wherein the photosynthetic microorganism has been modified to reduce fatty acid synthesis. In particular, such methods may be useful for upregulating synthesis of, or for production and/or isolation of, acetyl-CoA, acetate, butyrate, acetaldehyde, ethanol or acetyl phosphate.

As used herein, “fatty acid” refers to molecules a carboxylic acid (—COOH) with an aliphatic hydrocarbon chain. “Fatty acids” include salts and ions of fatty acids. For example, the fatty acid “butyric acid” includes the free acid butyric acid as well as butyrate, etc. “Short-chain” fatty acid as used herein, unless otherwise stated, refers to fatty acids having a 2-8 carbon chain length. Short-chain fatty acids may be 2, 3, 4, 5, 6, 7, or 8 carbons in chain length, for example 2-7, 2-6, 2-5, 2-4, 2-3 or 2 carbons in length. “Long-chain” fatty acids refers to those fatty acids which have longer chains than short-chain fatty acids. For example, long-chain fatty acids may refer to those a chain length of 13 or greater, preferably a chain length of 13-21, 13-20, 13-19, 13-18, 13-17, 13-16, 13-15, 13-14, 14-21, 14-20, 14-19, 14-18, 14-17, 14-16, 14-15, 14, 15-21, 15-20, 15-19, 15-18, 15-17, 15-16, 15, 16-21, 16-20, 16-19, 16-18, 16-17, 16, 17-21, 17-20, 17-19, 17-18, 17, 18-21, 18-20, 18-19, 18, 19-21, 19-20, 19, 20-21, 20, or 21 carbons.

Fatty acid synthesis could be inhibited in a number of ways. The photosynthetic microorganism could be modified to prevent the entry of acetyl-CoA into fatty acid biosynthesis pathways, or specific enzymes of fatty acid biosynthesis could be targeted for knockdown or knockout.

In some embodiments, the photosynthetic microorganism has been modified to reduce the conversion of acetyl-CoA to malonyl-ACP.

In some embodiments, the photosynthetic microorganism has been modified to reduce acyl-acyl carrier protein synthase activity.

In some embodiments, the photosynthetic microorganism has been modified to reduce or knock out native acyl-acyl carrier protein synthase (AAS) activity, e.g. to reduce or knock out expression or activity of an enzyme having AAS activity and having at least 80% sequence identity (optionally one of 85%, 90%, 91%, 92%, 93%, 94%, 95%, 96%, 97%, 98%, 99% or 100% sequence identity (to the amino acid sequence of GenBank accession number BAA17024.1.

Synechocystis AAS is known as the rate-controlling reaction in fatty acid biosynthesis by catalysing the condensation of malonyl-acyl carrier protein (malonyl-ACP) and acetyl-ACP. The amino acid sequence of AAS of Synechocystis sp. PCC 6803 has the GenBank accession number BAA17024.1.

In some embodiments, the photosynthetic microorganism has been modified to increase the conversion of butyryl-ACP to butyric acid.

In some embodiments, the photosynthetic microorganism has been modified to increase acyl-ACP-thioesterase expression.

Acyl-ACP-thioesterase expression could be increased through the heterologous expression of an acyl-ACP-thioesterase expression from organisms such as Anaerococcus tetradius (GenBank ID: EEI82564), Bacteroides fragilis (GenBank ID: CAH09236) and Haemophilus influenzae (GenBank ID: AAC22485.1), or any other suitable host.

In some embodiments, the photosynthetic microorganism has been modified to heterologously express the thioesterase gene Tes4 from Bacteroides fragilis (GenBank ID: CAH09236).

In some embodiments, the photosynthetic microorganism has been modified to reduce or knock out the native acyl-acyl carrier protein synthase activity, and to heterologously express an acyl-ACP-thioesterase gene (e.g. Tes4 from Bacteroides fragilis).

In some embodiments, the photosynthetic microorganism has been modified to increase the carbon flux from pyruvate to ethanol.

Carbon flux from pyruvate to ethanol could be increased through reducing carbon flux to competing pathways, by increasing photosynthetic efficiency, by improving enzymatic activity or by any other suitable method.

As shown in FIG. 2, one method of modifying a photosynthetic microorganism to reduce fatty acid synthesis is to reduce or knock out the native acyl-acyl carrier protein synthase activity, and/or to heterologously express an acyl-ACP-thioesterase gene.

In the embodiment shown in FIG. 2, the native acyl-acyl carrier protein synthase gene (aas) is knocked out. By knocking out this gene, carbon flux towards ethanol is increased. This change in carbon flux is achieved as a result of a reduction in the conversion of acetyl-CoA to malonyl-ACP. This increases ethanol synthesis and reduces fatty acid synthesis. Knocking out aas also reduces the conversion of butyric acid to butyryl-ACP.

In the embodiment shown in FIG. 2, Tes4 from Bacteroides fragilis is also heterologously expressed. This expression of Tes4 reduces the levels of butyryl- and longer chain fatty acyl-ACP molecules by generating the equivalent free fatty acid.

Methods of Modifying Photosynthetic Microorganisms

Photosynthetic microorganisms can be modified in a number of different ways. They can be modified to increase or decrease the expression of a native gene, and they can also be modified to express a heterologous gene.

The expression of native genes can be reduced through insertional mutagenesis, CRISPR gene editing and through other methods known in the art (Behler et al., 2018; Eungrasamee et al., 2019).

The expression of a heterologous gene or polypeptide can be induced by introducing the gene into a host cell via a vector. The vector may be used to replicate the nucleic acid in a compatible host cell. Therefore, nucleic acids can be produced by introducing a polynucleotide into a replicable vector, introducing the vector into a compatible host cell and growing the host cell under conditions that bring about replication of the vector.

A “vector” as used herein is an oligonucleotide molecule (DNA or RNA) used as a vehicle to transfer foreign genetic material into a cell. The vector may be an expression vector for expression of the foreign genetic material in the cell. Such vectors may include a promoter and/or a ribosome binding site (RBS) sequence operably linked to the nucleotide sequence encoding the sequence to be expressed. A vector may also include a termination codon and expression enhancers. Such expression vectors are routinely constructed in the art of molecular biology and may for example involve the use of plasmid DNA and appropriate initiators, promoters, RBS, enhancers and other elements, such as for example polyadenylation signals, which may be necessary and which are positioned in the correct orientation in order to allow for protein expression.

Any suitable vectors, promoters, enhancers and termination codons known in the art may be used to express a polypeptide from a vector according to the invention. In some embodiments, the vector may be a plasmid, phage, MAC, virus, etc.

In some embodiments the vector may be a prokaryotic expression vector, e.g. a bacterial expression vector.

In some embodiments, the vector may be a eukaryotic expression vector. In some embodiments, the vector may be a eukaryotic expression vector, e.g. a vector comprising the elements necessary for expression of protein from the vector in a eukaryotic cell. In some embodiments, the vector may be a mammalian expression vector, e.g. comprising a cytomegalovirus (CMV) or SV40 promoter to drive protein expression. In some embodiments, the vector may be a microalgal expression vector, e.g. pCB740.

In some embodiments, the vector may be a chloroplast expression vector, to enable transgene integration in the chloroplast genome.

Other suitable vectors would be apparent to persons skilled in the art. By way of further example in this regard we refer to Sambrook et al., 2001, Molecular Cloning: a laboratory manual, 3^{rd} edition, Cold Harbour Laboratory Press.

The term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. The resulting transcript may then be translated into a desired peptide or polypeptide. The promoter may be a T7 promoter.

In some embodiments, the vector may comprise element for facilitating translation of encoded protein from mRNA transcribed from the construct. For example, the construct may comprise a ribosomal binding site (RBS) such as a Shine-Dalgamo (SD) sequence upstream of the start codon.

In some embodiments, the vector may encode one or more response elements for modulating expression of the encoded protein(s). In some embodiments, the response element is an element that causes upregulation of gene or protein expression in response to treatment with a particular agent. For example, the agent may induce transcription of DNA encoding the protein(s) from a vector including a response element for the agent. In some embodiments the agent may be cobalt (II) nitrate hexahydrate, and the vector may comprise the cobalt-inducible P_{coa} system. Other induction agent/response element combinations are known in the art.

In some embodiments, the vector may encode one or more response elements for constitutive expression of the encoded protein(s), such that no induction is necessary.

In some embodiments the vector may comprise a transcription terminator sequence downstream of the sequences encoding to the protein or proteins of interest. In some embodiments the terminator may be a T7 terminator sequence. In some embodiments the vector may comprise a sequence encoding a detectable marker in-frame with the sequence encoding the protein of interest to facilitate detection of expression of the protein, and/or purification or isolation of the protein (e.g. a His, (e.g. 6×His), Myc, GST, MBP, FLAG, HA, E, or Biotin tag, optionally at the N- or C-terminus).

Some methods of of genetic modification require the introduction of genetic material into the host cell. The nucleic acids/expression vectors can be introduced into a cell by any suitable means, which are well known to the skilled person. In some embodiments the nucleic acids/expression vectors are introduced into a cell by transformation, transduction, conjugation, transfection or electroporation.

In Vitro Culture

Methods according to the present invention may be performed, or products may be present, in vitro. The term “in vitro” is intended to encompass experiments with materials, biological substances, cells and/or tissues in laboratory conditions or in culture (including laboratory or industrial scale culture), whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

Sequence Identity

Pairwise and multiple sequence alignment for the purpose of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

### Materials and Methods

All chemicals and solvents were purchased from commercial suppliers, and were of analytical grade or better. Media components were obtained from Formedium (Norfolk, UK). DNA sequencing and oligonucleotide synthesis were performed by Eurofins MWG (Ebersberg, Germany). The mounted Hi-Power warm white LEDs and LED drivers were from Thorlabs (New Jersey, USA). The photobioreactor was a thermostatic flat panel FMT 150 (400 mL; Photon Systems Instruments, Czech Republic) with integral culture monitoring (OD 680/720 nm), pH and feeding control and an LED blue light panel (465-470 nm; maximum PPFD=1648 μE photons). Proton NMR was performed on a 400 mHz Bruker in deuterated chloroform.

Synechocystis Constructs Generation

The gene encoding thioesterase Tes4 from Bacteroides fragilis (UniProt: P0ADA1) was obtained from plasmid pET-TPC4 (Kallio et al., 2014), and ligated into blunt pJET1.2 plasmid. Tes4 was further sub cloned into the erythromycin resistant RSF1010 plasmid using the Biopart Assembly Standard for Idempotent Cloning (BASIC) method was used as described previously. Gene expression was controlled using the cobalt-inducible P_{coa} system (plasmid plY845: P_{coa}:tes4 [Distributed Biomanufacturing of Liquefied Petroleum Gas, Hoeven et al May 2019, bioRxiv 640474; doi: https://doi.org/10.1101/640474]). Plasmid assembly was validated by DNA sequencing.

Plasmids were transformed into the E. coli helper/cargo strain (100 μL; E. coil HB101 strain carrying the pRL623 and RSF1010 plasmids), conjugal strain (E. coil ED8654 strain carrying pRL443 plasmid) and Synechocystis sp. PCC 6803 Δaas strain (OD₇₃₀˜1) using the tri-parental conjugation method described previously (Yunus and Jones, 2018; Yunus et al., 2018) Each strain had been pre-treated by washing with Luria broth and BG11-Co medium for E. coli and Synechocystis, respectively, to remove the antibiotics (Yunus and Jones, 2018; Yunus et al., 2018). The mixture was incubated for 2 h (30° C., 60 μmol photons m⁻²s⁻¹), and then spread onto BG11 agar plates without antibiotic, and incubated for 2 d (30° C., 60 μmol m⁻²s⁻¹). Cells were scraped from the agar plate, re-suspended in 500 μL of BG11-Co medium, and transferred onto a new agar plate containing 20 μg/ml erythromycin. Cells were allowed to grow for one week until colonies appeared.

Synechocystis Batch Culture Growth

Photosynthetic Synechocystis growth was performed in BG11 medium using a modified protocol as follows: Initial cultures in BG11 medium were incubated at 30° C. under 30 μE white light until OD 720 nm reached 1.0 (˜4 days). Replicate culture aliquots (2 mL) were harvested by centrifugation and re-suspended in 1 mL BG11 medium supplemented with sodium bicarbonate (150 mM), inducer cobalt (II) nitrate hexahydrate (100 μM), 50 μg/ml kanamycin and 20 μg/ml erythromycin at 30° C. Cultures were sealed within 4 mL gas tight vials and incubated at 30° C. for 24-48 h under blue light (average 63 μE).

Cell-free culture supernatant samples (10 μL) were analysed for ethanol and butyrate content by HPLC using an Agilent Hi-Plex H column.

Synechocystis Fermentation

The photobioreactor (400 mL) was set up in batch mode with starter culture diluted 3:1 in fresh BG11+ medium (BG11 pH 8.0 containing TES buffer and 1 g/L sodium thiosulphate) in the presence of 150 mM NaHCO₃. Both pH control and CO₂ supply was maintained using 1M NaHCO₃ in 2×BG11+. The culture was maintained at 30° C. with maximal stirring with an airflow rate of 1.21 L/min, illumination of warm white light (30 μE), automated pH maintenance (1M acetic acid in 2×BG11⁺) and optical density monitoring (680 nm and 720 nm). After reaching an optical density of ˜0.5 (720 nm), cobalt (II) nitrate hexahydrate (100 μM) was added as required, the warm white illumination was increased to 60 μE the integral actinic blue LED light panel was activated to provide 500-750 μE blue light (460-480 nm). The culture was maintained at 30° C. for 18-48 hours, fed and not fed respectively, with manual HPLC sampling from the culture to quantify ethanol and butyrate levels.

Analytical Techniques

Aqueous culture metabolites (ethanol and butyric acid) were analysed by HPLC using an Agilent 1260 Infinity HPLC with a 1260 ALS auto sampler, TCC SL column heater and a 1260 refractive index detector (RID). Cell-free culture supernatant samples (10 μL injection) were analysed isocratically on an Agilent Hi-Plex H column (300×7.7 mm; 5 mM H₂SO₄) at 60° C. with a flow rate of 0.7 mL/min for 40 minutes. Analyte concentrations were calculated by comparing the peak areas to a standard curve generated under the same HPLC conditions.

### Example 2

### Results and Discussion

Ethanol Tolerance

Ethanol tolerance of wild-type, Δaas knock out and the plY845 strain was assessed. Synechocystis sp. PCC 6803 was designated as wild type, the Δaas strain had native acyl-acyl carrier protein synthase activity knocked out, and the plY845 strain expressed thioesterase Tes4 from Bacteroides fragilis (UniProt: P0ADA1) and had native acyl-acyl carrier protein synthase activity knocked out.

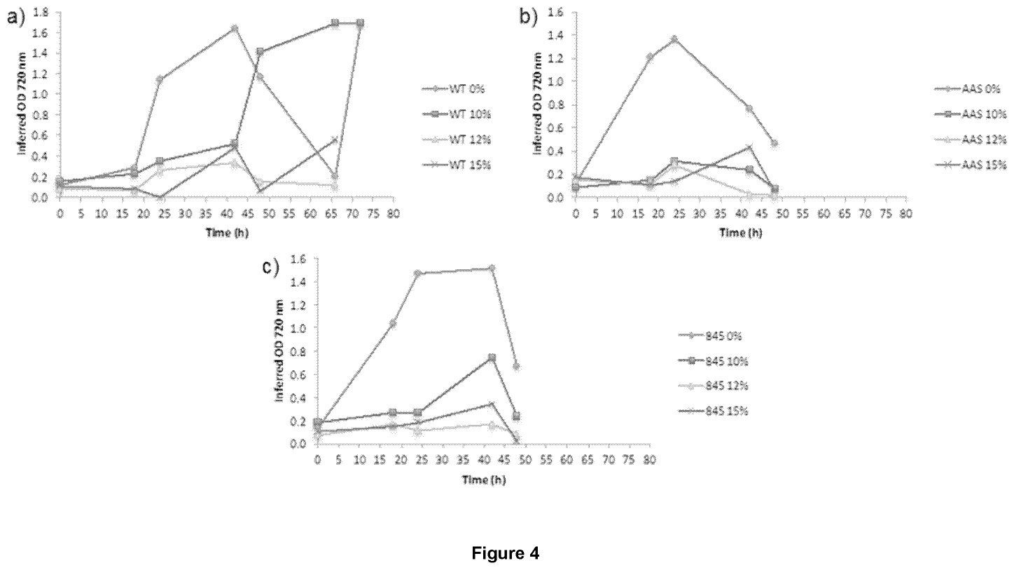

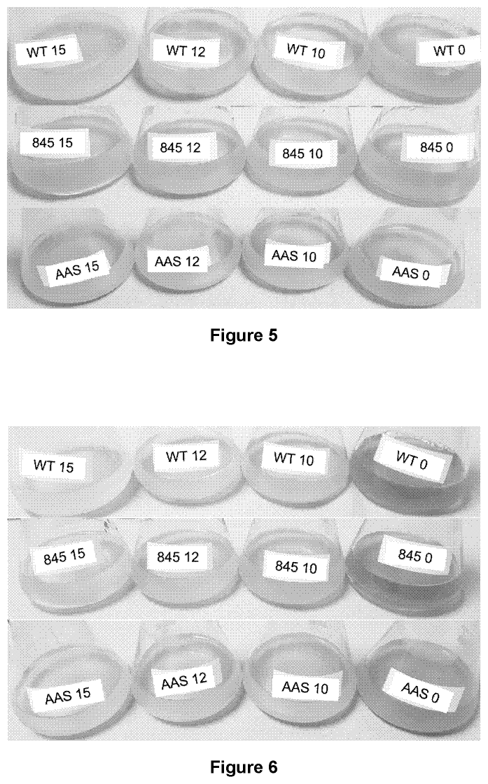

A viable industrial process where the ethanol is recovered and purified by distillation requires titres of at least ˜10% (w/v) ethanol (Katzen et al., 2019, Ruffing and Trahan, 2014). Lower than this and the cost of the heat required for stripping the ethanol from solution is higher than the value of the ethanol. The ethanol tolerance of Synechocystis was determined (0-15% (v/v)) with wild-type, Δaas knock out and the plY845 production strain, the latter of which expresses Tes4. Cell viability was confirmed by culturing a small aliquot of liquid culture on a BG11⁺ plate and detecting visible growth after 48 hours. Cultures in 10% ethanol showed reduced growth compared to no added ethanol, but the plY845 strain still contained viable cells after 48 h (FIGS. 4-6). Cultures grown in the presence of 10-12% added ethanol grew poorly, with no viable cells after 24 h.

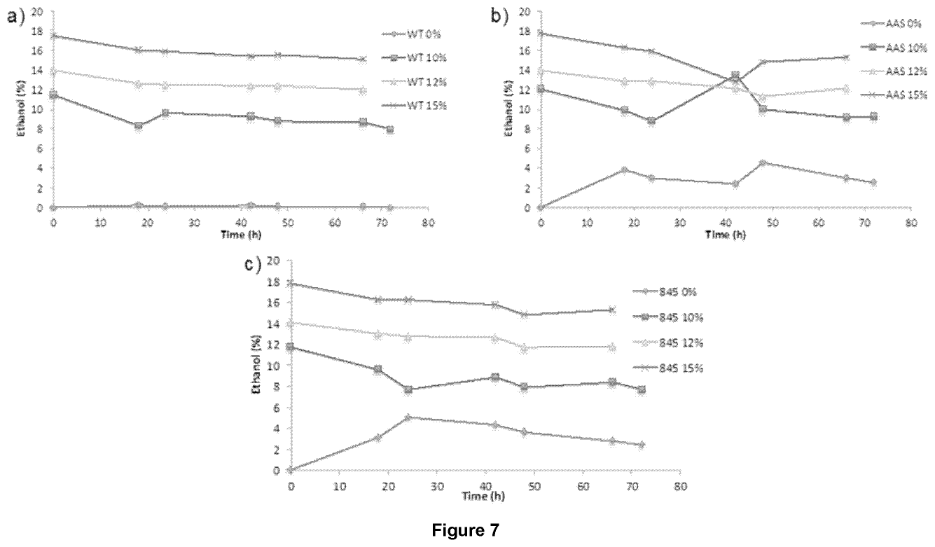

Given that additional ethanol would be produced during the growth, the actual concentration of ethanol was determined by HPLC (FIG. 7). This data shows around 2% extra ethanol was detected at the beginning of the growth, likely a carry-over from the starter culture. In addition, ethanol production was evident in the plY845 and Δaas strains where no ethanol had been added. Overall, cell viability is reduced in the presence of ethanol, but growth can still occur slowly at the critical 10% ethanol threshold. For the plY845 strain, growth in 10% ethanol is possible up to 48 h. Previous studies have shown that wild-type Synechocystis sp. 6803 can tolerate and grow well in 0.5 M ethanol (0.5 M˜2.3% w:v), and cells are viable long term up to 1 M ethanol (1M˜4.6% w:v) (Ruffing and Trahan, 2014).

Lab Scale Photobioreactor Production in White Light

Under standard photosynthetic conditions, it has previously been suggested that ethanol titres were approaching maximum theoretical values due to the limitations of the natural flux through pyruvate in Synechocystis 6803 (Yoshikawa et al., 2017, Dexter et al., 2015). We examined this by performing a small-scale (400 mL) culture of Synechocystis plY845 strain in a flatbed photobioreactor (Photon Systems Instruments) with continuous aeration, optical density monitoring (680 and 720 nm), pH and temperature control. Growth was performed in white light (30-60 μmol m⁻²s⁻¹) with air sparging (1.2 L/min, ˜3 culture volumes per min) and an initial NaHCO₃ addition (150 mM) as the CO₂ source. Manual sampling was performed to determine the concentration of secreted ethanol in the culture supernatant by HPLC.

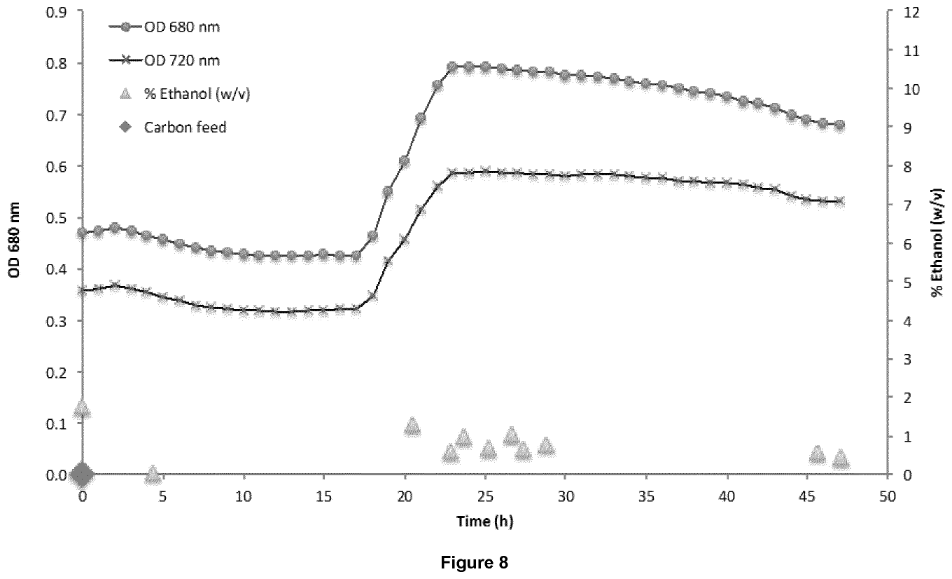

Overall the ethanol production levels were relatively low, generating ˜1.3% ethanol when exposed to standard photosynthetic conditions (FIG. 8). Ethanol production did not continue beyond 24 hours, presumably when the initial NaHCO₃ had all been consumed. The levels of carbon could be limiting growth and ethanol production; therefore it was surmised that a higher CO₂ supply is needed.

Effect of Blue Light and Bicarbonate Supplementation

The next stage was to perform a modified cultivation of Synechocystis plY845 under blue light with additional carbon supply and pH buffering by the addition of 150 mM NaHCO₃ at 0 and 43 h. These are not standard photosynthetic conditions as excessive blue light is known to cause photo bleaching of the carotenoid pigments, and gaseous CO₂ is usually the carbon source. In this cultivation the culture was exposed to blue light after 18 h to 800 μmol m⁻²s⁻¹ (or μE). This was decreased to 500 μE shortly thereafter when signs of photo bleaching were apparent. Manual sampling was performed to determine the concentration of secreted ethanol in the culture supernatant by HPLC.

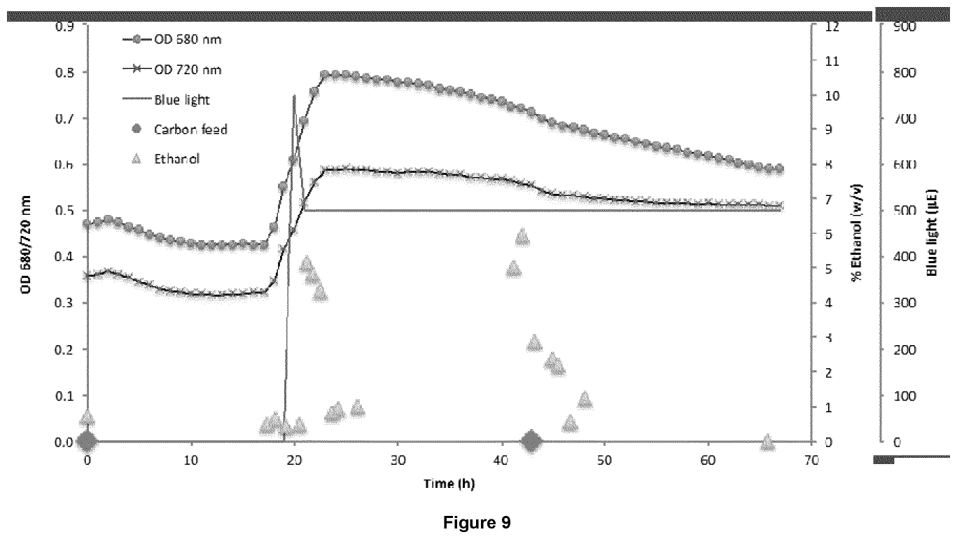

Ethanol concentrations fluctuated during the fermentation, however a maximum of 6% (w/v) was detected. This equated to a productivity of ˜1-2 g/L/h over the whole culture period, with 3-5 g/L/h during blue light illumination (FIG. 9). These titres are likely to be an underestimation as an unknown quantity of ethanol was lost due to incomplete condensation of gaseous ethanol escaping through the gas outlet. The presence of ethanol was confirmed by NMR. Culture growth declined once the highest level of ethanol was obtained, with minor photo bleaching apparent.

Confirmation of Ethanol Production

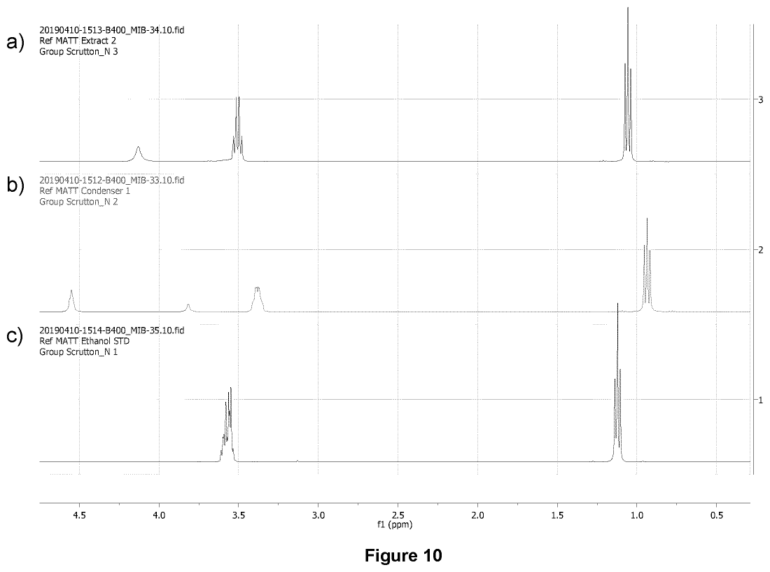

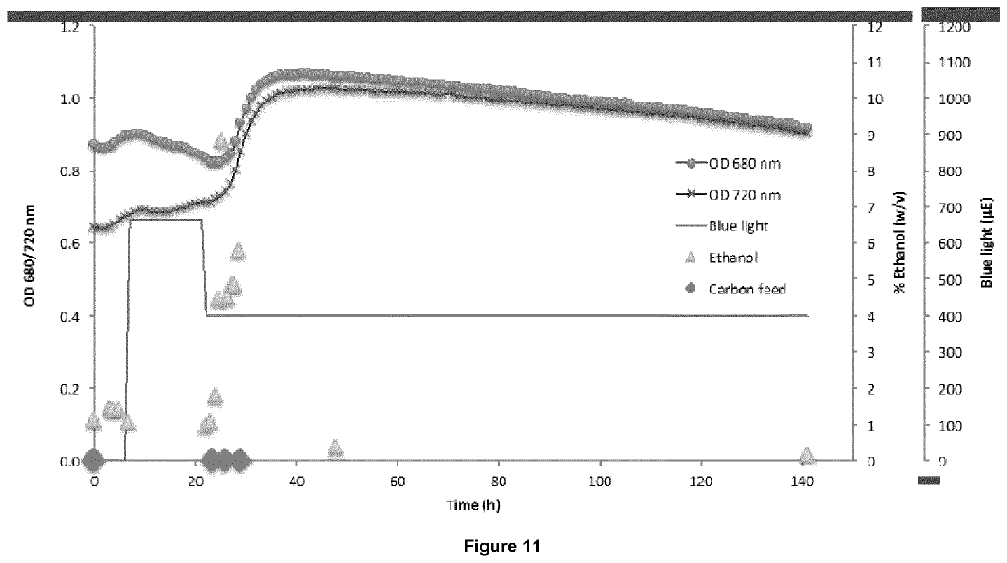

Initial ethanol detection was performed by HPLC, and compared to the retention time and standard curve of authenticated standards. To positively identify the product, we performed proton NMR on culture supernatant samples and condensate from the photobioreactor gas outlet condenser extracted into deuterated chloroform, and compared them to an authentic ethanol standard. Both the culture and condensate samples showed the same characteristic triplet (f1˜1.2) and doublet of quartet peaks (f1˜3.6) corresponding to an authenticated ethanol standard (FIG. 10). This suggests that the major constituent of our samples and the dominant species in the peak observed by HPLC was ethanol, as no other significant peaks were determined.

Effect of Initial Cell Density on Ethanol Production

As ethanol decreases the growth rate of Synechocystis plY845, an additional cultivation of the plY845 strain was performed as above with blue light and NaHCO₃ supplementation (500 mM at 23, 26 and 29 h), however the initial cell density was higher (OD 680 nm=0.8) prior to Tes4 expression. In this case the peak ethanol production increased to nearly 9% around 24 h, followed by a decline in cell density (FIG. 11). Given the known growth inhibition of Synechocystis in ethanol, a suitable strategy to maximise ethanol production could be to grow an uninduced culture to a high cell density, followed by induction and a relatively short cultivation time thereafter (48 h).

Effect of Carbon Limitation on Ethanol Production

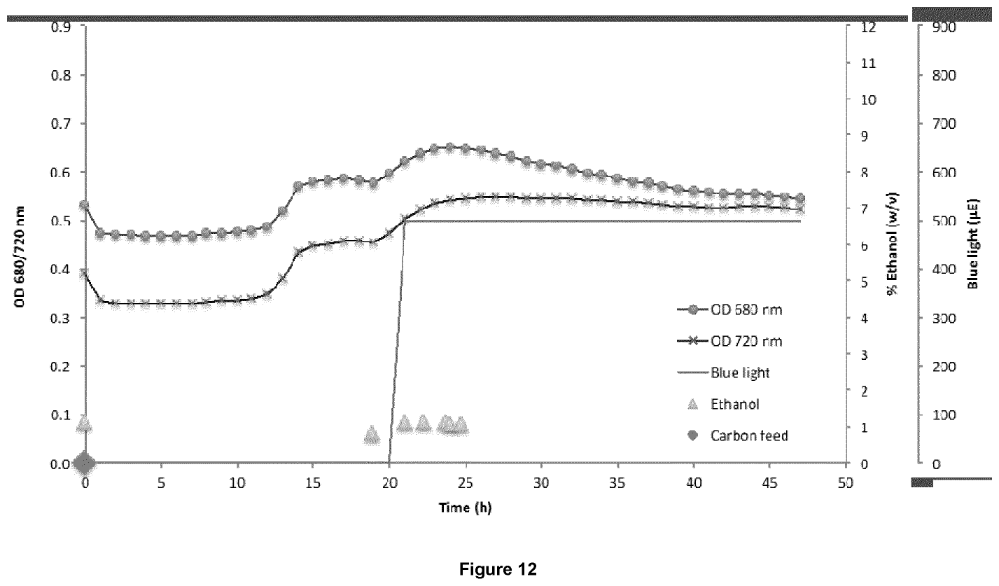

The effect of bicarbonate supplementation on ethanol production by Synechocystis plY845 under blue light was investigated to see if both blue light and high carbon (NaHCO₃) are required to obtain high ethanol titres. As only atmospheric CO₂ levels (280 ppm) were bubbled through the culture, the initial culture medium was supplemented with 150 mM NaHCO₃ as the carbon source. No further additions of NaHCO₃ were added, which will likely lead to carbon limitation during the later stages of growth. The cultivation under reduced bicarbonate supplementation showed a significant (6-fold) decrease in ethanol titres (˜1% (v/v); FIG. 12) compared to cultures grown with additional carbon feeds. Photo bleaching was apparent after ˜24 h, and the blue light intensity was decreased to minimise this effect.

Advantages in growth rates, carbon fixation rates and product titres have previously been reported when using an abundant carbon sink (Oliver and Atsumi, 2015). Carbon limitations could also affect the pH buffering of the culture, which may lead to a greater susceptibility to photo bleaching and lower tolerance to high intensity blue light. Additional pH buffering was supplied by the addition of TES buffer and automated pH control was implemented to minimise this effect in all cultures. However NaHCO₃ supplementation may increase tolerance to high intensity blue light by allowing a more rapid carbon fixation, providing a carbon sink for the extra photosynthetically derived electrons.

Effect of Blue Light on Ethanol Concentrations

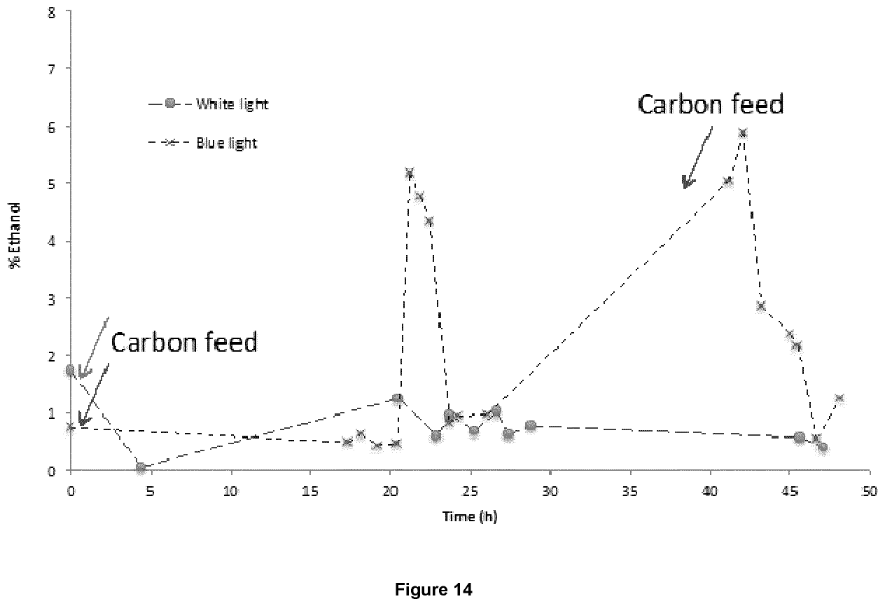

Comparative data between white and blue light is seen with the plY845 strain (FIG. 14) under controlled conditions in the photobioreactor (controlled pH, temperature and CO₂ supply with continuous monitoring of culture growth). Results clearly show that the blue light upregulates ethanol synthesis, and also suggest that pyruvate synthesis is also upregulated to lead to the increase in ethanol production, as there is no known alternative pathway to produce ethanol in Synechocystis 6803.

Important points to note with this data are the importance of frequent CO₂ (bicarbonate) addition and the switching of white to blue light. Initial growth was performed under white light to increase the cell density, followed by Tes4 induction. The earlier burst of ethanol production seen under blue light is not maintained as the carbon supply is limiting under these conditions, and ethanol is lost due to constant aeration. The later burst of ethanol under blue light is due to feeding in more carbon. This suggests the potential of ethanol production under blue light is significantly higher than we have detected due to carbon limitation, and this gives rise to high predicted yields when grown under aeration with a waste industrial CO₂ rich gas supply, effectively capturing this CO₂ as ethanol loss by evaporation and biomass.

Overall low levels of ethanol production are seen continuously under white light, showing blue light generates more ethanol than white light. Note that there is 1-2% ethanol at the start of the cultivation due to production of the starter culture under batch culture conditions. It is likely that under white light the ethanol production is a similar rate to loss by evaporation.

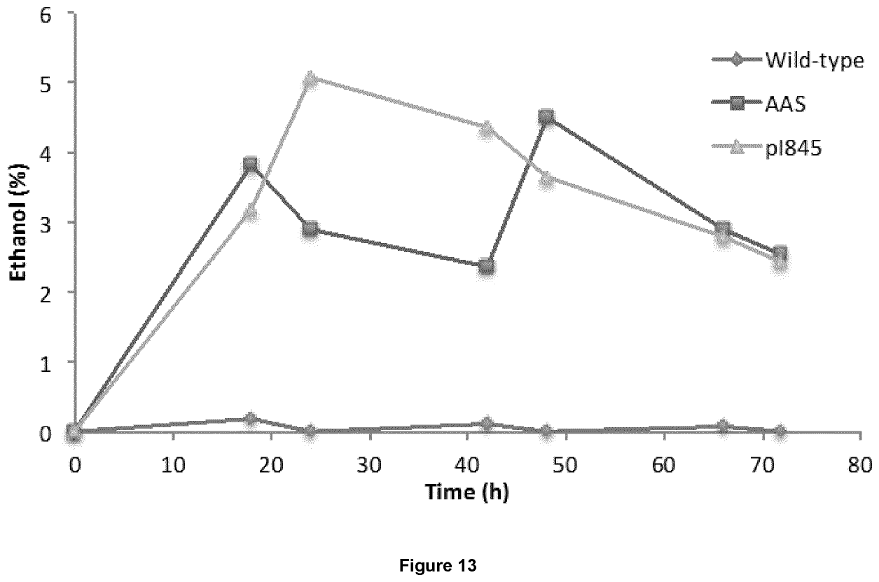

The overall levels are lower than expected for both white and blue light samples (FIG. 13), as under photobioreactor conditions there is constant aeration (1.2 L/min) causing the ethanol to evaporate, and some of it is lost through the condenser.

Effect of Genetic Changes on Ethanol Concentrations

As can be seen in FIG. 13, the modifications of the Synechocystis cultures have led to a dramatic increase in ethanol titres (˜28-fold) under normal white light conditions in batch (flask) cultures. Therefore the two changes in the genetic makeup of Synechocystis (Tes4 expression and aas knockout) make a significant contribution to increasing titres, even in the absence of high intensity blue light.

Discussion

Results show increased ethanol productivity and titres over previously reported studies for any cyanobacterial-based cultivation method (Luan et al., 2015, Gao et al., 2012, Yoshikawa et al., 2017). Results also demonstrate that high intensity blue light can be utilised to increase the growth rate, the biomass yield and the ethanol produced by both Synechocystis wild type and our production strain plY845.

The metabolic engineering of Synechocystis 6803 to increase flux through pyruvate and acetyl-CoA allows a greater efficiency of conversion from environmental CO₂ to secreted products such as ethanol. The combination of mutations made in the production strain and the blue light growth conditions synergistically result in even higher ethanol titres and productivity than any factor individually.

These results indicate that pyruvate synthesis has been upregulated by the blue light conditions. The route to ethanol production in Synechocystis requires pyruvate production as an intermediate step. It therefore follows that any route in Synechocystis that leads to increasing ethanol titres must by definition involve increasing carbon flux through pyruvate.

### REFERENCES

A number of publications are cited above in order to more fully describe and disclose the invention and the state of the art to which the invention pertains. Full citations for these references are provided below. The entirety of each of these references is incorporated herein.

1 Lehtinen, T., Virtanen, H., Santala, S. & Santala, V. Production of alkanes from CO₂ by engineered bacteria. Biotechnol. Biofuels 11, 228, (2018).

2 Luan, G. et al. Combinatory strategy for characterizing and understanding the ethanol synthesis pathway in cyanobacteria cell factories. Biotechnol. Biofuels 8, 184, (2015).

3 Mokashi, K., Shetty, V., George, S. A. & Sibi, G. Sodium bicarbonate as inorganic carbon source for higher biomass and lipid production integrated carbon capture in Chlorella vulgaris. Achiev. Life Sci. 10, 111-117, (2016).

4 Gao, Z., Zhao, H., Li, Z., Tan, X. & Lu, X. Photosynthetic production of ethanol from carbon dioxide in genetically engineered cyanobacteria. Energy Environ Sci 5, 9857-9865, (2012).

5 Kanno, M., Carroll, A. L. & Atsumi, S. Global metabolic rewiring for improved CO₂ fixation and chemical production in cyanobacteria. Nat Commun 8, 14724, (2017).

6 Yoshikawa, K., Toya, Y. & Shimizu, H. Metabolic engineering of Synechocystis sp. PCC 6803 for enhanced ethanol production based on flux balance analysis. Bioprocess Biosyst. Eng. 40, 791-796, (2017).

7 Lawrence, A. D. et al. Solution structure of a bacterial microcompartment targeting peptide and its application in the construction of an ethanol bioreactor. ACS Synth. Biol. 3, (2014).

8 Carrieri, D. et al. Boosting autofermentation rates and product yields with sodium stress cycling: Application to production of renewable fuels by cyanobacteria. Appl Environ Microbiol 76, 6455-6462, (2010).

9 Oliver, J. W. K. & Atsumi, S. A carbon sink pathway increases carbon productivity in cyanobacteria. Metab Eng 29, 106-112, (2015).

10 Subashchandrabose, S. R., Ramakrishnan, B., Megharaj, M., Venkateswarlu, K. & Naidu, R. Consortia of cyanobacteria/microalgae and bacteria: Biotechnological potential. Biotechnol. Adv. 29, 896-907, (2011).

11 Dexter, J., Armshaw, P., Sheahan, C. & Pembroke, J. T. The state of autotrophic ethanol production in cyanobacteria. J. Appl. Microbiol. 119, 11-24, (2015).

12 Mohd Azhar, S. H. et al. Yeasts in sustainable bioethanol production: A review. Biochem. Biophys. Rep. 10, 52-61, (2017).

13 Hirosawa, T. (ed Horst Senger) 39-47 (Springer Berlin Heidelberg).

14 Luimstra, V. M. et al. Blue light reduces photosynthetic efficiency of cyanobacteria through an imbalance between photosystems I and II. Photosynth Res 138, 177-189, (2018).

15 Vidal, R., López-Maury, L., Guerrero, M. G. & Florencio, F. J. Characterization of an alcohol dehydrogenase from the cyanobacterium Synechocystis sp. strain PCC 6803 that responds to environmental stress conditions via the Hik34-Rre1 two-component system. J. Bacteriol. 191, 4383-4391, (2009).

16 Nogales, J., Gudmundsson, S. & Thiele, I. Toward systems metabolic engineering in cyanobacteria: opportunities and bottlenecks. Bioengineered 4, 158-163, (2013).

17 von Berlepsch, S. The acyl-acyl carrier protein synthetase from Synechocystis sp. PCC 6803 mediates fatty acid import. Plant Physiol. 159, 606-617, (2012).

18 Kallio, P., Pásztor, A., Thiel, K., Akhtar, M. K. & Jones, P. R. An engineered pathway for the biosynthesis of renewable propane. Nat Commun 5, 4731, (2014).

19 Volker, A. R. et al. Fermentative production of short-chain fatty acids in Escherichia coli. Microbiology 160, 1513-1522, (2014).

20 Klähn, S. et al. Alkane biosynthesis genes in cyanobacteria and their transcriptional organization. Front. Bioeng. Biotechnol. 2, 1-12, (2014).

21 Lea-Smith, D. J. et al. Contribution of cyanobacterial alkane production to the ocean hydrocarbon cycle. Proc. Natl. Acad. Sci. 112, 13591-13596, (2015).

22 Sorigué, D. et al. Microalgae synthesize hydrocarbons from long-chain fatty acids via a light-dependent pathway. Plant Physiol 171, 2393-2405, (2016).

23 Dienst, D. et al. Transcriptomic response to prolonged ethanol production in the cyanobacterium Synechocystis sp. PCC6803. Biotechnol. Biofuels 7, 21, (2014).

24 Held, W. A., Ballou, B., Mizushima, S. & Nomura, M. Assembly mapping of 30 S ribosomal proteins from Escherichia coli further studies. J. Biol. Chem. 249, 3103-3111, (1974).

25 Shen, G. & Vermaas, W. F. Chlorophyll in a Synechocystis sp. PCC 6803 mutant without photosystem I and photosystem II core complexes. Evidence for peripheral antenna chlorophylls in cyanobacteria. J. Biol. Chem. 269, 13904-13910, (1994).

26 Kwon, J.-H., Bernát, G., Wagner, H., Rögner, M. & Rexroth, S. Reduced light-harvesting antenna: Consequences on cyanobacterial metabolism and photosynthetic productivity. Algal Res. 2, 188-195, (2013).

27 Lea-Smith, D. J. et al. Phycobilisome-deficient strains of Synechocystis sp. PCC 6803 have reduced size and require carbon-limiting conditions to exhibit enhanced productivity. Plant Physiol. 165, 705-714, (2014).

28 Wilson, A. et al. A soluble carotenoid protein involved in phycobilisome-related energy dissipation in cyanobacteria. Plant Cell 18, 992-1007, (2006).

29 Sun, Y. et al. Light modulates the biosynthesis and organization of cyanobacterial carbon fixation machinery through photosynthetic electron flow. Plant Physiol. 171, 530-541, (2016).

30 Knoop, H. et al. Flux balance analysis of cyanobacterial metabolism: The metabolic network of Synechocystis sp. PCC 6803. PLOS Comput. Biol. 9, e1003081, (2013).

31 Nakajima, T. et al. Integrated metabolic flux and omics analysis of Synechocystis sp. PCC 6803 under mixotrophic and photoheterotrophic conditions. Plant Cell Physiol. 55, 1605-1612, (2014).

32 Nakajima, T., Yoshikawa, K., Toya, Y., Matsuda, F. & Shimizu, H. Metabolic flux analysis of the Synechocystis sp. PCC 6803 ΔnrtABCD mutant reveals a mechanism for metabolic adaptation to nitrogen-limited conditions. Plant Cell Physiol. 58, 537-545, (2017).

33 You, L., He, L. & Tang, Y. J. Photoheterotrophic fluxome in Synechocystis sp. strain PCC 6803 and its implications for cyanobacterial bioenergetics. J. Bacteriol. 197, 943-950, (2015).

34 Yunus, I. S. & Jones, P. R. Photosynthesis-dependant biosynthesis of medium chain-length fatty acids and alcohols. Metabolic Engineering 44, 81-88, (2018).

35 Yunus, I. S. et al. Synthetic metabolic pathways for photobiological conversion of CO₂ into hydrocarbon fuel. Metabolic Engineering 49, 201-211, (2018).

36 Rippka, R., Deruelles, J., Waterbury, J. B., Herdman, M. & Stanier, R. Y. Generic assignments, strain histories and properties of pure cultures of cyanobacteria. Microbiology 111, 1-61, (1979).

37 Katzen, R., Madson, P. W. & Moon, G. D. in The alcohol textbook (eds W. M. Ingledew, D. R. Kelsall, G. D. Austin, & C. Kluspies) 292-297 (Nottingham University Publishers, Nottingham, UK, 2019).

38 Ruffing, A. M. & Trahan, C. A. Biofuel toxicity and mechanisms of biofuel tolerance in three model cyanobacteria. Algal Res. 5, 121-132, (2014).

39 Oren A (September 2004). “A proposal for further integration of the cyanobacteria under the Bacteriological Code”. International Journal of Systematic and Evolutionary Microbiology. 54 (Pt 5): 1895-902. doi:10.1099/ijs.0.03008-0.

40. Gao, X., Sun, T., Pei, G., Chen, L. & Zhang, W. Cyanobacterial chassis engineering for enhancing production of biofuels and chemicals. Appl Microbiol Biotechnol 100, 3401-3413, (2016).

41. Fernandes B. D., Mota A, Teixeira J. A., Vicente A. A. Continuous cultivation of photosynthetic microorganisms: Approaches, applications and future trends. Biotechnol Adv. 2015 Nov. 1; 33(6 Pt 2):1228-45. doi: 10.1016/j.biotechadv.2015.03.004

42 Pors Y., Wüstenberg A., Ehwald R. A batch culture method for microalgae and cyanobacteria with CO₂ supply through polyethylene membranes. Journal of Phycology. 2010. https://doi.org/10.1111/j.1529-8817.2010.00844.x

43. Eungrasamee K., Miao R., Incharoensakdi A, Lindblad P. and Jantaro S. Improved lipid production via fatty acid biosynthesis and free fatty acid recycling in engineered Synechocystis sp. PCC 6803. Biotechnology for Biofuels 2019 12:8 https://doi.org/10.1186/s13068-018-1349-8.

44. Behler J., Vijay D., Wolfgang R. Hess W. R., Akhtar M. K. CRISPR-based technologies for metabolic engineering in cyanobacteria. Trends in Biotechnology 2018 36:10 https://doi.org/10.1016/j.tibtech.2018.05.011.

45. Ruffing A. M. Engineered cyanobacteria: Engineered cyanobacteria: Teaching an old bug new tricks, Bioengineered Bugs, 2011, 2:3, 136-149, DOI: 10.4161/bbug.2.3.15285

46. Pattanaik B. and Lindberg P., Terpenoids and their biosynthesis in cyanobacteria, Life 2015, 5, 269-293, doi:10.3390/life5010269.

47 Young J. D., Shastri A. A., Stephanopoulos G., Morgan J. A., Mapping photoautotrophic metabolism with isotopically nonstationary (13)C flux analysis. Metab Eng. 2011 November; 13(6):656-65. doi: 10.1016/j.ymben.2011.08.002.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A method of upregulating the synthesis of pyruvate and/or a metabolite of pyruvate in a photosynthetic microorganism, the method comprising culturing the photosynthetic microorganism under conditions suitable for photosynthesis wherein the culture is irradiated with light having a wavelength between 380 nm and 500 nm.

2. The method of claim 1, further comprising the step of isolating said pyruvate or metabolite of pyruvate from the photosynthetic microorganism.

3. The method of claim 1 or 2, wherein the metabolite of pyruvate is selected from one of ethanol, acetyl-CoA, alanine, butanol, butyrate, propane, acetate, or lactate.

4. A method for the photosynthetic production of a one, two, three or four carbon compound, the method comprising culturing a photosynthetic microorganism under conditions suitable for photosynthesis wherein the culture is irradiated with light having a wavelength between 380 nm and 500 nm, and isolating the one, two, three or four carbon compound.

5. The method of claim 4, wherein the one, two, three or four carbon compound is pyruvate or a metabolite of pyruvate.

6. The method of claim 5, wherein the metabolite of pyruvate is selected from one of ethanol, acetyl-CoA, alanine, butanol, butyrate, propane, acetate, or lactate.

7. The method of any one of the previous claims, wherein the culture is irradiated with light having a proportion of light having a wavelength between 380 nm and 500 nm that is greater than 35%.

8. The method of any one of the previous claims, wherein the culture is irradiated with light having a proportion of light having a wavelength between 501 nm and 800 nm that is less than 50%.

9. The method of any one of the previous claims, wherein said light having a wavelength between 380 nm and 500 nm has a photon dose greater than 50 μmol m⁻²s⁻¹, and optionally less than 1000 μmol m⁻²s⁻¹.

10. The method of any one of the previous claims, wherein the culture is irradiated with light having a wavelength between 380 nm and 500 nm for at least 5 minutes.

11. The method of any one of the previous claims, wherein the culture is irradiated with light having a wavelength between 380 nm and 500 nm for at least 60 minutes.

12. The method of any one of the previous claims, wherein the culture is irradiated with light from a light source that emits light having a proportion of light having a wavelength between 380 nm and 500 nm that is greater than 35%.

13. The method of any one of the previous claims, wherein the culture is irradiated with light from a light source that emits said light having a wavelength between 380 nm and 500 nm at a photon dose greater than 50 μmol m⁻²s⁻¹, and optionally less than 1000 μmol m⁻²s⁻¹.

14. The method of any one of the previous claims, wherein the culture is irradiated with light having a wavelength between 380 nm and 500 nm for a period of time sufficient to produce ethanol at a concentration of at least 2% (w/v) in the culture.

15. The method of any one of the previous claims, wherein the culture is irradiated with light having a wavelength between 380 nm and 500 nm for a period of time sufficient to produce ethanol at a concentration of at least 5% (w/v) in the culture.

16. The method of any one of the previous claims, wherein the culture is irradiated with light having a wavelength between 380 nm and 500 nm for a period of time sufficient to produce ethanol at over 0.1 g/L/h.

17. The method of any one of the previous claims, wherein the photosynthetic microorganism is cultured under white light irradiation for a period of time during which the photosynthetic microorganism may grow before light conditions are changed to irradiation with light having a wavelength between 380 nm and 500 nm.

18. The method of any one of the previous claims, wherein the pyruvate or metabolite of pyruvate is isolated by distillation.

19. The method of any one of the previous claims, wherein the photosynthetic microorganism has been modified to reduce fatty acid synthesis.

20. A method for the production of ethanol from a photosynthetic microorganism, the method comprising culturing the photosynthetic microorganism under conditions suitable for photosynthesis, wherein the photosynthetic microorganism has been modified to reduce fatty acid synthesis.

21. The method of claim 20, wherein the photosynthetic microorganism has been modified to (i) increase the carbon flux from pyruvate to ethanol and/or (ii) reduce the conversion of acetyl-CoA to malonyl-ACP.

22. The method of claim 20 or 21, wherein acyl-acyl carrier protein synthase activity is reduced or knocked out in the photosynthetic microorganism.

23. The method of any one of claims 20 to 22, wherein the photosynthetic microorganism has been modified to:
a) increase the conversion of butyryl-ACP to butyric acid; and/or
b) increase acyl-ACP-thioesterase expression; and/or
c) heterologously express an acyl-ACP-thioesterase gene.

24. The method of any previous claim, comprising a step wherein the culture is irradiated with a single peak of blue light.

25. The method of any previous claim, comprising a step wherein the culture is irradiated with a single peak of light having a wavelength between 380 nm and 500 nm.

26. The method of any previous claim, comprising a step wherein the culture is irradiated with light which comprises less than 30% red light, orange light, yellow light and/or green light.

27. The method of any previous claim, comprising a step wherein the culture is irradiated with light which comprises no red light.
